(19) United States
(12) Patent Application Publication
(10) Pub. No.: US 2011/0081403 A1
(43) Pub. Date: Apr. 7, 2011

(54) HISTONE OCTAMERS FOR INCREASED NUCLEIC ACID TRANSFER

(75) Inventor: Nancy Smyth Templeton, Houston, TX (US)

(73) Assignee: GRADALIS, INC., Carrollton, TX (US)

(21) Appl. No.: 12/887,666

(22) Filed: Sep. 22, 2010

Related U.S. Application Data
(60) Provisional application No. 61/247,729, filed on Oct. 1, 2009.

(51) Int. Cl. A61K 9/127; A61K 38/16; A61P 35/00

(52) U.S. Cl. 424/450; 514/19.3

(57) ABSTRACT

The present invention provides reconstituted histone octamers with multiple modifications (e.g. acetylation of all histones and trimethylation of histone H3K4) assembled onto plasmids for increased transcription post-transfection using our unique bi-lamellar invaginated liposomes (BIVs) to more effectively recruit the transcriptional machinery of human cancer cells post-transfection and substantially increase the production of therapeutic gene products.

### CROSS-REFERENCE TO RELATED APPLICATIONS

This application claims priority to U.S. Provisional Application Ser. No. 61/247,729 filed Oct. 1, 2009, the entire contents of which are incorporated herein by reference.

### TECHNICAL FIELD OF THE INVENTION

The present invention relates in general to increasing plasmid DNA-based expression that is sustained. It also relates to compositions and methods for transferring nucleic acids into cells, specifically to compositions and methods for delivery and providing nucleic acid transfer complexes that transfect cells with high efficiency.

### STATEMENT OF FEDERALLY FUNDED RESEARCH

None.

### BACKGROUND OF THE INVENTION

Without limiting the scope of the invention, its background is described in connection with nucleic acid transfer complexes using reconstituted histone octamers containing the appropriate modifications for transcriptional activation.

For example, U.S. Pat. No. 7,192,605, entitled Nucleic acid transfer complexes disclose compositions and methods for transferring nucleic acids into cells in vitro and in vivo and in preferred embodiments, the compositions comprise delivery systems providing nucleic acid transfer complexes that transfect cells with high efficiency.

Non-viral delivery is a desirable replacement for viral vectors due to numerous problems caused when virus-based vectors are used clinically; however, retaining positive aspects of viral delivery including robust expression of encoded therapeutic products is also desirable. Unlike viruses and viral vectors, non-viral plasmids cannot commandeer the transcriptional machinery after cell entry to produce their encoded gene products exclusively. Although other groups have used single, unmodified histones for the purpose of increasing nuclear delivery of plasmids, the increase in gene expression levels produced were modest and unacceptable for clinical uses.

### SUMMARY OF THE INVENTION

The present invention provides reconstituted histone octamers with multiple modifications (e.g. acetylation of all histones and trimethylation of histone H3K4) assembled onto plasmids for increased transcription post-transfection using our unique bi-lamellar invaginated liposomes (BIVs) to more effectively recruit the transcriptional machinery of human cancer cells post-transfection and substantially increase the production of therapeutic gene products.

The present inventors identified histone modifications by chromatin immunoprecipitation analyses on Keratin 8, the most highly expressed gene in the human breast cancer cell line, MCF-7, based on serial analysis of gene expression. Quantitative comparisons to the “normal” counterpart cell line, MCF-10A, expressing 350-fold lower levels of Keratin 8 and other breast cancer cell lines expressing higher levels were performed using real-time PCR. Extraordinarily high levels of trimethyl histone H3 lysine 4 (H3K4) were found primarily in the first intron of the Keratin 8 gene stretching from 400 to 2000 bp downstream from the promoter in all breast cancer cells lines but not in MCF-10A cells. The highest levels of histone H3K4 trimethylation in MCF-7 cells ranged from 70% to 80% over input within 1200 bp of this region. Knockdown of mixed-lineage leukemia (MLL), the specific methyltransferase for histone H3K4, with MLL-specific siRNA decreased histone H3K4 trimethylation on the Keratin 8 gene and decreased Keratin 8 mRNA levels. Histone H3K4 trimethylation mediates approximately 86% of the elevated, sustained expression of the Keratin 8 gene in MCF-7 cells.

Gene therapy clinical trials for cancer frequently produce inconsistent results. Some of this variability could result from differences in transcriptional regulation that limit expression of therapeutic genes in specific cancers. Systemic liposomal delivery of a nonviral plasmid DNA showed efficacy in animal models for several cancers. However, we observed large differences in the levels of gene expression from a cytomegalovirus CMV promoter-enhancer between lung and breast cancers. To optimize gene expression in breast cancer cells in vitro and in vivo, we created a new promoter-enhancer chimera to regulate gene expression. Serial analyses of gene expression data from a panel of breast carcinomas and normal breast cells predicted that the glyceraldehyde-3-phosphate dehydrogenase (GAPDH) promoter is highly active in breast cancers. Furthermore, GAPDH is up-regulated by hypoxia, which is common in tumors. We added the GAPDH promoter, including the hypoxia enhancer sequences, to our in vivo gene expression plasmid. The novel CMV-GAPDH promoter-enhancer showed up to 70-fold increased gene expression in breast tumors compared to the optimized CMV promoter-enhancer alone. No significant increase in gene expression was observed in other tissues. These data demonstrate tissue-specific effects on gene expression after nonviral delivery and suggest that gene delivery systems may require plasmid modifications for the treatment of different tumor types. Furthermore, expression profiling can facilitate the design of optimal expression plasmids for use in specific cancers.

### BRIEF DESCRIPTION OF THE DRAWINGS

For a more complete understanding of the features and advantages of the present invention, reference is now made to the detailed description of the invention along with the accompanying figures and in which:

FIG. 1 is a graph that compares histone modifications examined by ChIP within the Keratin 8 gene locus in MCF-7 cells.

FIG. 2 is a graph that compares novel plasmids the present invention by in vitro transfection of MCF7 breast cancer cells.

FIGS. 3A to 3C are graphs that compare in vitro transfection of various lung cancer and breast cancer cells using the novel, high expression plasmid pCAT-8. FIG. 3A compares pCAT-8 versus pCAT-4 in transfection of different breast cancer cells including T-47D, MCF7, SK-BR-3, and HCC1428. FIG. 3B compares pCAT-8 versus pCAT-4 in transfection of different lung cancer cells including H358, H460, H1299, and A549. FIG. 3C shows the means of the results shown in (FIG. 3A) and (FIG. 3B) were compared for fold increased CAT production using pCAT-8 versus pCAT-4 for each breast and lung cancer cell line. The control is set at onefold, which indicates no increased CAT production.

FIGS. 4A to 4B are graphs that compare in vitro transfection of MCF7 cells grown under reduced levels of oxygen post transfection using the novel, high-expression plasmid pCAT-8.

FIGS. 5A to 5B are graphs that compares In vitro transfection of MCF7 cells harvested at various time points post transfection using the novel, high-expression plasmid pCAT-8.

FIGS. 6A to 6C are graphs that compare gene expression in breast tumors in vivo using pCAT-8.

FIGS. 7A to 7B are graphs that compare gene expression in immune-competent, normal mice.

FIG. 8 is a graph of the Keratin 8 mRNA levels in MCF-7, SK-BR-3, T-47D, and MCF-10A cells.

FIG. 9 is a graph of Histone H3 K4 trimethylation within the Keratin 8 gene in MCF-7 and MCF-10A cells.

FIGS. 10A to 10F are graphs of the histone modifications (anti-acetyl H4, anti-acetyl H2B, anti-acetyl H3 K9, anti-dimethyl H3 K9, normal rabbit Ig and a Summary for MCF-7) within the Keratin 8 gene locus in MCF-7 and MCF-10A cells.

FIGS. 11A to 11B are graphs of the Histone H3 K4 trimethylation within the Keratin 8 gene in SK-BR-3 and T-47D cells.

FIGS. 12A to 12C are graphs of the effect of MLL knockdown on Keratin 8 mRNA levels in MCF-7, SK-BR-3, T-47D, and MCF-10A cells.

FIGS. 13A to 13D are graphs of the effect of MLL knockdown on Histone H3 K4 trimethylation within the Keratin 8 gene in MCF-7, SK-BR-3, T-47D, and MCF-10A cells.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The specific embodiments discussed herein are merely illustrative of specific ways to make and use the invention and do not delimit the scope of the invention.

To facilitate the understanding of this invention, a number of terms are defined below. Terms defined herein have meanings as commonly understood by a person of ordinary skill in the areas relevant to the present invention. Terms such as “a”, “an” and “the” are not intended to refer to only a singular entity, but include the general class of which a specific example may be used for illustration. The terminology herein is used to describe specific embodiments of the invention, but their usage does not delimit the invention, except as outlined in the claims.

As used herein the term “nucleic acid” denotes a polymer containing at least two nucleotides. “Nucleotides” contain a sugar deoxyribose (DNA) or ribose (RNA), a base, and a phosphate group. Nucleotides are the monomeric units of nucleic acid polymers. Nucleotides are linked together through the phosphate groups to form nucleic acid. A “polynucleotide” is distinguished here from an “oligonucleotide” by containing more than 100 monomeric units; oligonucleotides contain from 2 to 100 nucleotides. “Bases” include purines and pyrimidines, which further include natural compounds adenine, thymine, guanine, cytosine, uracil, inosine, and other natural analogs, and synthetic derivatives of purines and pyrimidines, which include, but are not limited to, modifications which place new reactive groups such as, but not limited to, amines, alcohols, thiols, carboxylates, and alkylhalides.

The term nucleic acid encompasses sequences that include any of the known base analogs of DNA and RNA including, but not limited to, 4-acetylcytosine, 8-hydroxy-N-6-methyladenosine, aziridinylcytosine, pseudoisocytosine, 5-(carboxyhydroxylmethyl)uracil, 5-fluorouracil, 5-bromouracil, 5-carboxymethylaminomethyl-2-thiouracil, 5-carboxymethylaminomethyluracil, dihydrouracil, inosine, N6-isopentenyladenine, 1-methyladenine, 1-methylpseudouracil, 1-methylguanine, 1-methylinosine, 2,2-dimethylguanine, 2-methyladenine, 2-methylguanine, 3-methylcytosine, 5-methylcytosine, N6-methyladenine, 7-methylguanine, 5-methylaminomethyluracil, 5-methoxy-aminomethyl-2-thiouracil, beta-D-mannosylqueosine, 5′-methoxycarbonylmethyluracil, 5-methoxyuracil, 2-methylthio-N-6-isopentenyladenine, uracil-5-oxyacetic acid methylester, uracil-5-oxyacetic acid, oxybutoxosine, pseudouracil, queosine, 2-thiocytosine, 5-methyl-2thiouracil, 2-thiouracil, 4-thiouracil, 5-methyluracil, N-uracil-5-oxyacetic acid methylester, uracil-5-oxyacetic acid, pseudouracil, queosine, 2-thiocytosine, and 2,6-diaminopurine. As used herein the term “privileged” genes, denotes the 0.03% most highly expressed genes within any specific cell.

As used herein, nucleic acid may be single (“ssDNA”), double (“dsDNA”), triple (“tsDNA”), or quadruple (“gsDNA”) stranded DNA, and single stranded RNA (“RNA”) or double stranded RNA (“dsRNA”). Nucleic acids may be linear, circular, or have higher orders of topology (e.g., supercoiled plasmid DNA). DNA may be in the form of anti-sense, plasmid DNA, parts of a plasmid DNA, vectors, expression cassettes, chimeric sequences, chromosomal DNA, or derivatives of these groups. RNA may be in the form of oligonucleotide RNA, tRNA (transfer RNA), snRNA (small nuclear RNA), rRNA (ribosomal RNA), mRNA (messenger RNA), anti-sense RNA, (interfering) double stranded RNA, ribozymes, chimeric sequences, or derivatives of these groups.

As used herein, the term “expression cassette” denotes a natural or recombinantly produced nucleic acid molecule that is capable of expressing protein(s). A DNA expression cassette typically includes a promoter (allowing transcription initiation), and a sequence encoding one or more proteins. Optionally, the expression cassette may include transcriptional enhancers, non-coding sequences, splicing signals, transcription termination signals, and polyadenylation signals. An RNA expression cassette typically includes a translation initiation codon (allowing translation initiation), and a sequence encoding one or more proteins. Optionally, the expression cassette may include translation termination signals, a polyadenosine sequence, internal ribosome entry sites (IRES), and non-coding sequences.

As used herein, the term “gene” generally denotes a nucleic acid sequence that comprises coding sequences necessary for the production of a therapeutic nucleic acid or a polypeptide or precursor. The polypeptide can be encoded by a full length coding sequence or by any portion of the coding sequence so long as the desired activity or functional properties (e.g., enzymatic activity, ligand binding, signal transduction) of the full-length polypeptide or fragment are retained.

As used herein, the terms “nucleic acid molecule encoding,” “DNA sequence encoding,” and “DNA encoding” denote the order or sequence of deoxyribonucleotides along a strand of deoxyribonucleic acid. The order of these deoxyribonucleotides determines the order of amino acids along the polypeptide (protein) chain. The DNA sequence thus codes for the amino acid sequence. As used herein, the terms “an oligonucleotide having a nucleotide sequence encoding a gene,” “a polynucleotide having a nucleotide sequence encoding a gene,” and “a nucleic acid having a nucleotide sequence encoding a gene,” mean a nucleic acid sequence comprising the coding region of a gene or in other words the nucleic acid sequence which encodes a gene product. The coding region may be present in either a cDNA, genomic DNA or RNA form.

As used herein, the term “isolated” when referring to a nucleic acid or a peptide or polypeptide is a nucleic acid or the polypeptide that is essentially free from contaminating cellular components, such as carbohydrate, lipid, nucleic acid or proteinaceous impurities associated with the nucleic acid or the polypeptide in nature.

Typically, a preparation of an isolated nucleic acid or an isolated polypeptide is a nucleic acid or a polypeptide in a purified form, i.e., at least about 80% pure, at least about 90% pure, at least about 95% pure, greater than 95% pure, or greater than 99% pure. One way to show that a particular nucleic acid or protein preparation contains an isolated nucleic acid or polypeptide is by the appearance of a single band following agarose gel, polyacrylamide, sodium dodecyl sulfate (SDS)-polyacrylamide gel electrophoresis of the nucleic acid protein preparation and staining of the gel (e.g., ethidium bromide or Coomassie Brilliant Blue). However, the term “isolated” does not exclude the presence of the same nucleic acid or polypeptide in alternative physical forms, such as single stranded or double stranded nucleic acids or for proteins dimmers, trimers, tetramers, or alternatively, glycosylated, or other derivatized forms. Often the phrase “isolated and purified” will be used in certain contexts and it means that the component is isolated and purified away from the location where the component is found in nature, e.g., the isolation and purified of a plasmid away from a host cell by use of a mini-prep or equivalent procedure. For a protein, homogenization of cells typically yields the release of protein (although proteins can be isolated and purified from a supernatant).

As used herein, the term “complex” through a process called “complexation” or “complex formation,” denotes contact with one another through “non-covalent” interactions such as, but not limited to, electrostatic interactions, hydrogen bonding interactions, and hydrophobic interactions. An “interpolyelectrolyte complex” is a non-covalent interaction between polyelectrolytes of opposite charge. A molecule is “modified,” through a process called “modification,” by a second molecule if the two become bonded through a covalent bond. That is, the two molecules form a covalent bond between an atom form one molecule and an atom from the second molecule resulting in the formation of a new single molecule. A chemical “covalent bond” is an interaction, bond, between two atoms in which there is a sharing of electron density.

As used herein, the term “chromatin” denotes the nucleoprotein structure of the cellular genome. Cellular chromatin comprises nucleic acid, primarily DNA, and protein, including histones and non-histone chromosomal proteins. The majority of eukaryotic cellular chromatin exists in the form of nucleosomes of about 150 base pairs of DNA associated with an octamer of two each of histones H2A, H2B, H3 and H4; and linker extends between nucleosome cores. A molecule of histone H1 is generally associated with the linker DNA. For the purposes of the present disclosure, the term “chromatin” is meant to encompass all types of cellular nucleoprotein, both prokaryotic and eukaryotic and chromosomal and episomal chromatin.

The liposome fuses with the plasma membrane, thereby releasing the compound into the cytosol. Alternatively, the liposome is phagocytosed or taken up by the cell in a transport vesicle. Once in the endosome or phagosome, the liposome is either degraded or it fuses with the membrane of the transport vesicle and releases its contents. Liposomes are microscopic vesicles that comprise amphipathic molecules that contain both hydrophobic and hydrophilic regions. Liposomes can contain an aqueous volume that is entirely enclosed by a membrane composed of amphipathic molecules. Liposomes are formed from one to several different types of amphipathic molecules and methods have been developed to complex biologically active compounds with liposomes. Generally, liposomes can be divided into three groups based upon their overall size and lamellar structure. Small unilamellar vesicles about 20 to 30 nm in diameter and contain one single lipid bilayer surrounding the aqueous compartment. Multi-lamellar vesicles that contain multiple aqueous compartments and bilayers. Large uni-lamellar vesicles usually 150 to 200 nm in diameter. Efficient nucleic acid transfer in vitro has been accomplished with the use of positively-charged liposomes that contain cationic lipids. For example, the cationic lipid, N-1-(2,3dioleyloxy)propyl-N,N,N-trimethylammonium chloride (DOTMA), cholesterol, phosphatidylcholines, or phosphatidylserines, wherein the acyl group chain length is between 16 and 20. DOTMA was combined with dioleoylphosphatidylethanolamine (DOPE) to form liposomes that spontaneously complexed with nucleic acids. DNA or RNA entrapment occurs because the positively-charged liposomes naturally complex with negatively-charged nucleic acids. DNA has been shown to induce fusion of cationic liposomes containing DOTMA/DOPE. A variety of cationic lipids have been made in which a glycerol or cholesterol hydrophobic moiety is linked to a cationic headgroup by metabolically degradable ester bond. These have included 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB) and cetyltrimethylammonium bromide (CTAB), Stearylamine. A series of cationic, non-pH sensitive lipids that included DORI (1,2-dioleoyl-3-dimethyl-hydroxyethyl ammonium bromide), DORIE (1,2-dioleyloxypropyl-3-dimethyl-hydroxyethyl ammonium bromide), and DMRIE (1,2-dimyristyloxypropyl-3-dimethyl-hydroxyethyl ammonium bromide) have been reported and studied. Other non-pH-sensitive, cationic lipids include: O,O′-didodecyl-N-p-(2-trimethylammonioethyloxy)benzoyl-N,N,N-tri-methylammonium chloride, Lipospermine, DC-Chol (3a-N-(N′,N″-dimethylaminoethane)carbonylcholesterol), lipopoly(L-lysine), cationic multilamellar liposomes containing N-(a-trimethylammonioacetyl)-didodecyl-D-glutamate chloride (TMAG), TRANSFECTACE (1:2.5 (w:w) ratio of DDAB which is dimethyl dioctadecylammonium bromide and DOPE) (Life Technologies) and LIPOFECTAMINE (3:1 (w:w) ratio of DOSPA which is 2,3-dioleyloxy-N-20({2,5-bis(3-aminopropyl)amino-1-oxypentyl}amino)ethyl-N,N-dimethyl-2,3-bis(9-octad-ecenyloxy)-1-propanaminium trifluoroacetate and DOPE).

Another example of the preparation of liposome includes mixing chloroform or ethanol solutions of the different lipids in microcentrifuge tubes and removing the solvent by nitrogen gas flow to produce dried lipid films. One ml of sterile water or 10 mM HEPES buffer, pH 7.8, was added, and the tubes were sealed and vortexed for 1 min at room temperature and sonicated to obtain a clear emulsion. Compositions were stored at 4° C. A liposome formulation consisting of DOTAP, DOPE, and DLPE, in a ratio of 10:9:1 (w:w:w) or 14.85:12.11:1.73 (DOTAP:DOPE:DLPE).

The present invention provides reconstituted histone octamers containing the appropriate modifications for transcriptional activation, and thus more effectively recruit the transcriptional machinery of human cancer cells post-transfection. The present invention improved non-viral delivery with BIVs, reversible masking, targeted delivery using small molecules, and other inventions. We have also had success in Phase I clinical trials, including treatment of non-small lung cancer patients who have failed chemotherapy. We wish to further improve success by accomplishing high levels of sustained expression from DNA-based plasmids.

The present invention uses robust tissue-specific enhancers, histone deacetylase inhibitors (HDACs), parts of histones or histone octamers with a single modification (e.g. acetylation). Histones form an octamer of two H2A/H2B dimers and one H3/H4 tetramer around which 146 base pairs of DNA are wound to form a nucleosome. The N-terminal tails of histones can be post-translationally modified by acetylation, methylation, phosphorylation, ubiquitination, sumoylation, ADP-ribosylation, citrullination (deimination), and others. Some modifications such as acetylation of the histones and methylation of histone H3 lysine 4 (H3K4) are associated with transcriptional activation; whereas, lysine methylation of histones H3K9, K27, K79 and others are associated with transcriptional repression.

The present invention provides compositions that mimic key elements from “privileged genes” (e.g., the 0.03% most highly expressed genes within any specific cell determined by serial analysis of gene expression (SAGE)) in order to enhance gene expression in human MCF-7 orthotopic breast tumors. The present invention provides taking sequence elements from the privileged gene GAPDH, the 7^{th} highest expressed gene in MCF-7 cells, produced up to 70-fold increased gene expression specifically in tumors post-injections of BIV-chloramphenicol acetyltransferase (CAT) DNA complexes in MCF-7 tumor-bearing mice. The GAPDH sequences added to our CAT-plasmid (pCAT-4), downstream from the optimal CMV-promoter enhancer also contain a hypoxia enhancer with binding sites for HIF-1 proteins. This new plasmid is called pCAT-8. CAT production was measured by CAT ELISA (Roche), and normalized to total protein (Micro BCA; Pierce).

We also examined the Keratin 8 gene, the highest expressed gene in MCF-7 cells, for histone modifications by chromatin immunoprecipitation analyses (ChIP). We found widespread, extraordinarily high levels of H3K4 trimethylation (70%-80% over input) within the first intron of the Keratin 8 gene locus of MCF-7 cells and SK-BR-3 and T-47D breast cancer cells with high levels of Keratin 8 expression, and not in the normal cells that have 350-fold lower levels of Keratin 8 gene expression.

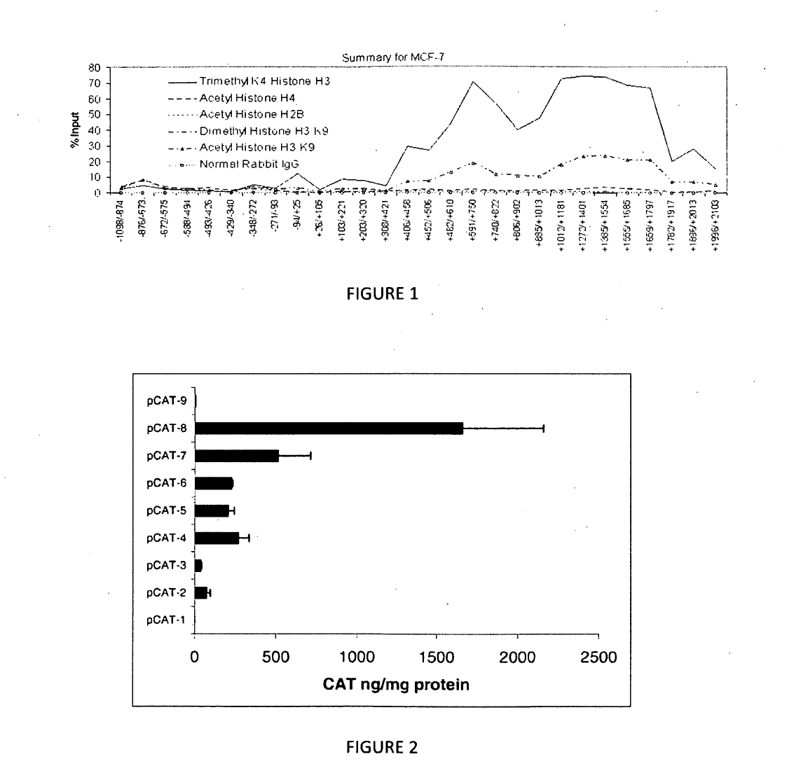

FIG. 1 is a graph of the summary and comparison of the histone modifications examined by ChIP within the Keratin 8 gene locus in MCF-7 cells. High levels of H3K4 trimethylation have been shown to recruit the transcriptional machinery to produce highly efficient transcription elongation versus stalled pol II transcription initiation complexes. Although the acetylation patterns of H4, H2B, and H3K9 did not significantly differ among the breast cancer and normal cells, acetylations are necessary but not sufficient for mediating privileged gene expression. Some investigators have also shown that acetylation of histone octamers increased transcription 15-fold by a pol III promoter. Interestingly, H3K4 trimethylation is required for human herpes simplex virus 1 (HSV-1) lytic infection. Specifically, H3K4 trimethylation of the virus genome is required for HSV-1 transcription and replication.

The present invention provides histone octamers with multiple modifications (e.g. acetylation of all histones and trimethylation of histone H3K4) that will be assembled onto plasmids for increased transcription post-transfection using BIVs [18]. Although other groups have used single, unmodified histones for the purpose of targeted delivery of plasmids to the nucleus, the increase in gene expression levels produced were modest, approximately 6-fold [19] and un acceptable for clinical uses. The present invention uses reconstituted histone octamers containing the appropriate modifications for robust transcriptional activation, and thus more effectively recruit the transcriptional machinery of human cancer cells post-transfection.

The present invention provides modified reconstituted histone octamers, e.g., recombinant human histones H2A, H2B, H3 and H4; modifying these histones by acetylation, and trimethylation of H3K4; creating H2A/H2B dimers and H3/H4 tetramers using the modified histones; and mixing these dimers and tetramers to form modified histone octamers.

The present invention provides plasmids associated with modified nucleosomes. The modified histone octamers assembled on negatively supercoiled plasmid DNA encoding reporter genes (e.g. CAT, luciferase) at different ratios of octamer:DNA with or without using salt gradient dialysis. The present invention provides plasmid DNA associated with modified nucleosomes encapsulated in BIVs. These liposomal complexes will be used to transfect different human cancer cell types versus normal cells in culture. The results will determine the best ratio of octamer:DNA to use. The use of modified nucleosomes will also be compared to using single modified histones or HDAC inhibitors.

The present invention provides reconstituted histone octamers H2A, H2B, H3 and H4 that can be used in clinical trials, and for transfections in human cancer cells and human tumors. However, the sequences of these core histones are highly conserved across different species. Briefly, these core human histones were produced large-scale in Escherichia coli and contain N-terminal, cleavable His6 tags for ease in purification. The His6 tags were removed using the thrombin protease whose active site of cleavage was included in the histones' amino acid sequences between the N-termini of the histones and the His6 tags. The N-termini of histone tails protrude away from the nucleosome and are available to be modified, e.g. by acetylation, methylation, and others stated above. Investigators have shown that individual histones can be acetylated by GCN5 first and then successfully assembled into octamers that were used for nucleosome reconstitution. For example, the histones can be acetylate using GCN5 and/or the p300 catalytic domain, and trimethylated H3K4 using Set9a histone methyltransferase specific to H3K4. Refolding and reconstitution of the H2A/H2B dimers and H3/H4 tetramers using the modified histones, and mixing these dimers and tetramers to form modified histone octamers will be performed using published procedures. Assessments of the products formed during the multi-step assembly will also be performed, including SDS-PAGE electrophoresis to verify the proper ratio of H2A:H2B:H3:H4. Investigators reported that nucleosomes form instantaneously in a temperature-independent manner when mixing histone octamers with negatively supercoiled DNA.

BIVs can efficiently encapsulate all types of nucleic acids, viruses, proteins, drugs, antibodies, peptides, and mixtures of these reagents. For example, we have successfully encapsulated a mixture of plasmid DNA and protein for vaccine studies. The present invention provides BIV-pCAT-8 complexes, our CAT plasmid with the GAPDH promoter-enhancer downstream from the optimized CMV promoter-enhancer, versus BIV-pCAT-8-MNP complexes in MCF-7 cells. Using pCAT-8 increased gene expression post-injection in orthotopic MCF-7 breast tumors in mice by up to 70-fold. For comparison, we will also perform all transfections listed above in MCF-10A cells, the “normal” counterpart breast epithelial cell line to MCF-7. MCF-10A cells are often used as the near normal control cell line because they are human mammary gland cells that have a normal or near-normal karyotype.

The present invention provides acetylated H2A, H2B, H4; and H3 that is both acetylated and trimethylated at K4 before assembling modified histone octamers. We will also mix each one of these individually modified histones with pCAT-4 or p-CAT-8 (e.g. pCAT-4+acetylatedH2A, pCAT-4+acetylatedH2B, and so forth) versus single unmodified histones to compare transfection levels using BIVs in MCF-7 and MCF-10A cells. HDAC inhibitors such as trichostatin A (TSA) and/or butyric acid have been used to increase transcription levels of non-viral vectors and viral vectors post-transfection in various cell types. The HDAC inhibitors, valproic acid and vorinostat, are also used in clinical trials to treat cancer by increasing the expression levels of genes required to induce cell cycle arrest and/or apoptosis. Cancer cell lines include and are not limited to melanoma (Sk-Mel-28, LOX IMVI, UACC-62), non-small cell lung (H460, H520, SK-Lu-1, H1299, A549), pancreatic (PANC1, miaPaCa2), liver, ovarian, prostate, cervical, colorectal (CCL-247), and other breast cancers (T-47D, MDA-MB-231, SK-BR-3).

Successful nonviral gene therapy requires optimization of several components, including the plasmid design, plasmid DNA preparation, delivery vehicle formulation, route of administration, detection of gene expression, dosing, and administration schedule [1]. Ultimately, for efficacy in gene replacement, the plasmid must express the cDNA of interest at adequate levels in the target cell. High levels of plasmid DNA can be consistently detected in the nucleus of transfected cells. However, in some cases the expression of the gene encoded by the plasmid in these cells remained low or undetectable [2]. This problem exists for plasmids and not for viral delivery vectors. Viral vectors encode viral proteins that could act in cis to up-regulate gene expression from promoters, e.g., the cytomegalovirus (CMV) promoter, and therefore are not limited by host cell transcriptional regulation.

Inconsistent clinical trial results from experimental cancer drugs and gene therapies have been attributed to the molecular heterogeneity of tumors as well as to differences in their pharmacodynamics [3]. In gene therapy, lack of efficacy in certain patients could be due to differences in transcriptional regulation between individual cancers. These differences could compromise both the expression of an introduced gene and the efficacy of its gene product. For example, the efficacy of apoptosis-inducing proteins requires transcriptional activation of specific signal transduction pathways to induce cell death [4]. Our data suggest that transcriptional heterogeneity may be responsible for the lack of gene expression in certain cancer cell types after nonviral plasmid DNA delivery. These variations in gene expression cannot be explained by differences in delivery of plasmid DNA to the nuclei by our novel liposomes for systemic gene delivery [5-9]. We have demonstrated efficacy of this delivery system in small and large animal models for lung [6], breast [9], head and neck (Hung and Templeton, unpublished data), and pancreatic cancers [7] and for hepatitis B and C (Clawson and Templeton, unpublished data). The liposomes of the present invention can be used to treat these cancers. A CMV promoter-enhancer has large differences in the levels of gene expression among several breast cancer lines.

Serial analyses of gene expression (SAGE) [11] data from specific breast tumors and cell lines indicated that the human glyceraldehyde-3-phosphate dehydrogenase (GAPDH) promoter is selectively and highly expressed in breast cancers. To optimize gene expression in breast cancers, the present invention provides a new promoter-enhancer chimera incorporating the GAPDH promoter. Furthermore, GAPDH is up-regulated during hypoxia [12], which is common in tumors. Therefore, we included the GAPDH sequences responsive to up-regulation during hypoxia (hypoxia enhancer) in our in vivo gene expression plasmid to improve its specificity further.

SAGE data in normal and tumor breast cells. SAGE provides an absolute quantitation of mRNA for every expressed known or unknown gene [11]. Therefore, this technology is more quantitative than microarray analyses, differential PCR, or subtractive hybridization for assessing gene expression. The present invention provides more efficient promoters and enhancers for use in treating breast tumor cells, and other tissues (e.g., brain, colon, endothelium, and prostate) and in breast cancer/DCIS versus normal breast using SAGEmap xProfiler (www.ncbi.nlm.nih.gov/SAGE/sagexpsetup.cgi). The presenters used both tissue and cell SAGE libraries for screening to identify genes that are highly expressed at the mRNA level both in the tumor cell lines use in xenograft models and in authentic human breast tumors. Table 1 lists several highly abundant and selectively expressed transcripts.

/For these candidate genes, we performed SAGE Virtual Northern analysis (www.ncbi.nlm.nih.gov/SAGE/sagevn.cgi) and SAGE gene to tag mapping (www.ncbi.nlm.nih.gov/SAGE/SAGEcid.cgi). These tools provided a method to query expression levels of specific genes in the entire database and suggested that the GAPDH, keratin-8, deoxythymidylate kinase, and ribosomal L30 gene promoters may be useful for efficient expression of transgenes in breast cancers as well as normal breast. The GAPDH gene transcripts were highly abundant in breast cancer cells, particularly in MCF7 cells, and were underrepresented in normal mammary epithelium (Table 1). The human GAPDH promoter has been characterized [15], and the sequences that up-regulate GAPDH expression during hypoxia (hypoxia enhancer) have also been identified [12].

TABLE 1
Summary of expression levels for selected genes identified by xProfiler (expression listed as SAGE tag frequency/10⁶)

| | Unigene ID Hs: | | | | | |
|---|---|---|---|---|---|---|
| | 79006 | | | | | 265827 |
| | Deoxythymidylate | 69855 | 242463 | 111222 | 169476 | IFNα-inducible |
| Cell/tissue | kinase | N-Ras, related | Keratin-8 | Ribosomal L30 | GAPDH | protein |
| MCF7 control 0 h | 9381 | 6267 | 12,522 | 9381 | 6958 | 3962 |
| MGF7 + estrogen 10 h | 9530 | 5807 | 12,492 | 9630 | 4351 | 2945 |
| Mammary epithelium | 1342 | 101 | 1,261 | 1342 | 549 | 40 |
| DCIS | 3783 | 0 | 600 | 3783 | 339 | 194 |
| HMEC-B41 | 3546 | 0 | 1,418 | 3546 | 6382 | 0 |
| MDA-453 | 1743 | 105 | 2,536 | 1743 | 951 | 158 |
| SK-BR-3 | 4660 | 490 | 6,623 | 4660 | 1962 | 0 |
| DCIS2 purified cells | 2215 | 173 | 1,523 | 2215 | 450 | 7338 |
| BrN normal luminar epithelial cells | 2582 | 79 | 718 | 2682 | 532 | 0 |

Table 2 lists several novel plasmids containing the GAPDH promoter-enhancer and compared them with existing plasmids to express the reporter gene chloramphenicol acetyltransferase (CAT) in MCF7 cells in culture. Nine different plasmids, designated pCAT-1 through pCAT-9 (Table 2), were used for transfections with extruded DOTAP liposomes prepared by a protocol previously developed in our laboratory [5].

TABLE 2
Different components of the plasmid constructs

| | |
|---|---|
| pCAT-1 | EF-1α promoter/enhancer → CAT gene → pEFIRES backbone |
| pCAT-2 | pVAX CMV promoter/enhancer → CAT gene → pVAX backbone |
| pCAT-3 | GAPDH promoter/enhancer → CAT gene → pVAX backbone |
| pCAT-4 | p4119 CMV promoter/enhancer → p4119 intron → CAT gene → pVAX backbone |
| pCAT-5 | GAPDH promoter/enhancer → p4119 CMV promoter/enhancer → p4119 intron → CAT gene → pVAX backbone |
| pCAT-6 | p4119 CMV promoter/enhancer → GAPDH promoter/enhancer → p4119 intron → CAT gene → pVAX backbone |
| pCAT-7 | p4119 CMV promoter/enhancer → p4119 intron → GAPDH promoter/enhancer → CAT gene → pVAX backbone |
| pCAT-8 | p4119 CMV promoter/enhancer → GAPDH promoter/enhancer → CAT gene → pVAX backbone |
| pCAT-9 | p4119 CMV promoter/enhancer → reversed GAPDH promoter/enhancer sequences → CAT gene → pVAX backbone |

Components are listed in the order found within each plasmid. Only the features that distiguish each plasmid are listed. The pVAX promoter/enhancer is short, consisting of 600 bp. The p4119 CMV promoter/enhancer is longer, consisting of 800 bp. The GAPD promoter/enhancer is 800 bp in length. The p4119 intron is 400 bp in length. The CAT gene from p4119 was used for all constructs.

These liposomes transfect a wide variety of cells in vitro ([8]; N. S. Templeton, unpublished data). The components that distinguish each plasmid are shown in Table 2 in the order found within each plasmid. Using the pEFIRES plasmid that contains only the elongation factor-1α promoter-enhancer to express the CAT gene, we constructed pCAT-1. In previous work, a pEFIRES-maspin cDNA construct was encapsulated in the extruded DOTAP:Chol liposomes developed in our laboratory [5]. These maspin DNA-liposome complexes demonstrated efficacy in a syngeneic breast tumor metastasis model after intravenous or direct tumor injections [9]. The mammary gland tumors were established using a PyV MT parental tumor cell line that was isolated from MMTV polyomavirus middle T transgenic mice. However, pCAT-1 produced no detectable levels of CAT production after transfection in MCF7 cells.

FIG. 2 is a graph of a comparison of novel plasmids by in vitro transfection of MCF7 breast cancer cells. MCF7 cells were transfected with the CAT reporter plasmids pCAT-1 through pCAT-9, described in Table 2. Extruded DOTAP-DNA liposome complexes were used for transfection. CAT production was normalized to protein concentration in the cell extracts and is presented as the mean±SD. pCAT-8, containing the optimized CMV promoter-enhancer 5′ of the GAPDH promoter-enhancer, produced the highest levels of CAT production. The pVAX plasmid (Invitrogen, Carlsbad, Calif.) contains a shorter length CMV promoter-enhancer of approximately 600 bp. However, this plasmid is useful because the pVAX backbone is minimized for bacterial sequences and contains a kanamycin resistance gene to use for antibiotic selection during plasmid growth. For use in preparing plasmid DNA for injection into humans in clinical trials, kanamycin selection is used and ampicillin selection is prohibited. The CAT-2 plasmid contains the CAT gene subcloned into pVAX, and it produced low levels of CAT after transfection in MCF7 cells. The pVAX CMV promoter-enhancer was removed from pCAT-2 and replaced with the GAPDH promoter-enhancer to produce pCAT-3. CAT production in MCF7 cells remained low using pCAT-3 for transfection.

Previously, we used a CAT plasmid, p4119, designed for in vivo gene delivery and gene expression [16,17]. This plasmid contains a longer length CMV promoter-enhancer of approximately 800 bp and an intron of 400 bp 5′ to the start codon of the CAT gene. However, the p4119 plasmid backbone is longer than that in pVAX, containing about 590 bp more bacterial sequences, and contains the ampicillin-resistance gene for selection. We constructed pCAT-4 by replacing the pVAX CMV promoter-enhancer in pCAT-2 with the p4119 CMV promoter-enhancer and intron. pCAT-4 produced 3.7-fold increased CAT production compared to pCAT-2 (FIG. 2, P<0.01). The plasmids pCAT-5, -6, and -7 are variations of pCAT-4 that contain the GAPDH promoter-enhancer in different locations (Table 2). pCAT-5 contains the GAPDH promoter-enhancer 5′ to the p4119 CMV promoter-enhancer. The pCAT-6 contains the GAPDH promoter-enhancer 3′ to the p4119 CMV promoter-enhancer and 5′ to the p4119 intron. pCAT-7 contains the GAPDH promoter-enhancer 5′ to the CAT gene. After transfection of MCF7 cells, only pCAT-7 produced 1.9-fold increased CAT production compared to pCAT-4 (P<0.025) and 7-fold increased CAT production compared to pCAT-2 (FIG. 2, P<0.025).

To improve pCAT-7, the p4119 intron was removed to bring the p4119 CMV promoter-enhancer closer to the GAPDH promoter-enhancer. This novel construct was named pCAT-8, and it produced the highest levels of CAT production after transfection of MCF7 cells (FIG. 1) at 1656.2 ng of CAT per milligram of total protein. Therefore, pCAT-8 produced 6.2-fold increased CAT production compared to pCAT-4 (P<0.05) and 22.5-fold increased CAT production compared to pCAT-2 (FIG. 1, P<0.05). By reversing the GAPDH promoter-enhancer sequences within pCAT-8 to create pCAT-9, CAT production dropped to nearly undetectable levels after transfection in MCF7 cells. These data suggest that the highest levels of increased gene expression and protein production in MCF7 cells were mediated by the GAPDH promoter-enhancer sequences within the plasmid. Other plasmids that contained deletions, with or without the hypoxia enhancer sequences, in the GAPDH promoter-enhancer region within pCAT-8 were tested. However, these plasmids produced reduced levels of CAT expression compared to pCAT-8 (data not shown). Our results comparing pCAT-8 versus pCAT-6 for transfection of MCF7 cells also showed that the 3′ position of the GAPDH promoter-enhancer is essential and it must be placed directly 5′ to the start of the CAT coding region.

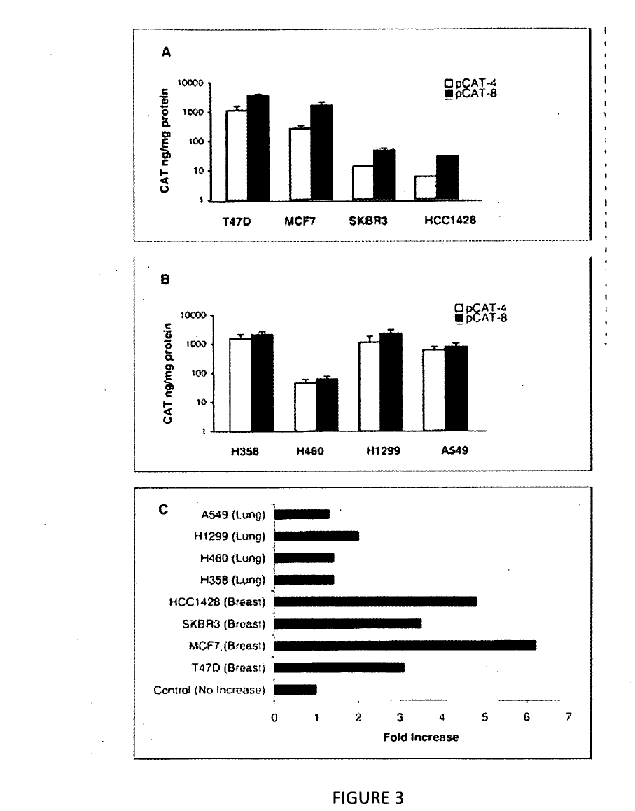

FIG. 3 is a graph of the In vitro transfection of various lung cancer and breast cancer cells using the novel, highexpression plasmid pCAT-8. Our “generic” in vivo CAT expression plasmid, pCAT-4, was compared to pCAT-8, which contains the optimized CMV promoter-enhancer 5′ to the GAPDH promoter-enhancer. (FIG. 3A) Comparison of pCAT-8 versus pCAT-4 in transfection of different breast cancer cells including T-47D, MCF7, SK-BR-3, and HCC1428. Extruded DOTAP-DNA liposome complexes were used for transfection. CAT production was normalized to protein concentration in the cell extracts and is presented as the mean±SD. (FIG. 3B) Comparison of pCAT-8 versus pCAT-4 in transfection of different lung cancer cells including H358, H460, H1299, and A549. Extruded DOTAP-DNA liposome complexes were used for transfection. CAT production was normalized to protein concentration in the cell extracts and is presented as the mean±SD. (FIG. 3C) The means of the results shown in (FIG. 3A) and (FIG. 3B) were compared for fold increased CAT production using pCAT-8 versus pCAT-4 for each breast and lung cancer cell line. The control is set at onefold, which indicates no increased CAT production.

In vitro transfection of various lung cancer and breast cancer cells using the novel plasmid. To determine any cell-type specificity of increased CAT production mediated by the GAPDH promoter-enhancer sequences, we transfected a variety of different breast cancer and lung cancer cells and compared the results. The breast cancer cell lines transfected were T-47D, MCF7, SK-BR-3, and HCC1428. The lung cancer cell lines transfected were H358, H460, H1299, and A549. All cells were transfected either with our generic in vivo CAT plasmid, pCAT-4, or with the novel CAT plasmid containing the GAPDH promoter-enhancer that produced the highest levels of CAT production in our initial studies, pCAT-8. FIG. 3C shows the fold increase in CAT production in each cell line after transfection with pCAT-8 versus pCAT-4. The control is 1-fold and indicates no increase. pCAT-8 increased CAT production in all breast cancer cells between 3.1- and 6.2-fold (P<0.01), whereas CAT production increased in all lung cancer cells from 1.3- to 2-fold (P<0.01) using pCAT-8 for transfection. These data show that the GAPDH promoter-enhancer improved CAT production after transfection significantly more in breast cancer cells than in lung cancer cells.

Overall gene expression in SK-BR-3 and HCC1428 breast cancer cells and H460 lung cancer cells, however, remained lower in comparison to the other cells. Other promoter-enhancer elements could be identified and used to increase gene expression further in these breast cancer or lung cancer cells. Therefore, the present invention provides custom gene expression cassettes engineered to treat specific subtypes of breast cancer cells, e.g., an MCF7 and T-47D subtype versus an SK-BR-3 and HCC1428 breast cancer cell subtype or an H460 subtype versus an H1299, A549, and H358 lung cancer subtype.

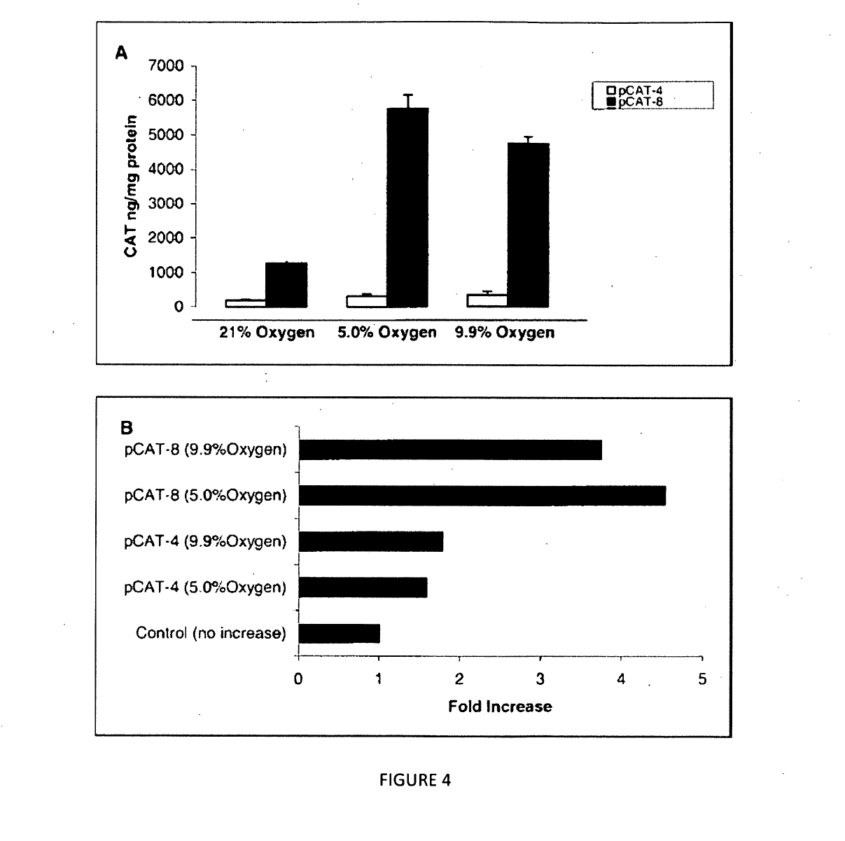

FIG. 4 is a graph of In vitro transfection of MCF7 cells grown under reduced levels of oxygen post transfection using the novel, high-expression plasmid pCAT-8. Our generic in vivo CAT expression plasmid, pCAT-4, was compared to pCAT-8, which contains the optimized CMV promoter-enhancer 5′ to the GAPDH promoter-enhancer. (FIG. 4A) Comparison of pCAT-8 versus pCAT-4 in transfection of MCF7 cells grown in culture chambers containing 21, 5.0, or 9.9% oxygen post transfection. Extruded DOTAP-DNA liposome complexes were used for transfection. CAT production was normalized to protein concentration in the cell extracts and is presented as the mean±SD. (FIG. 4B) The means of the results shown in (FIG. 4A) were compared for fold increased CAT production using pCAT-8 versus pCAT-4 for transfection of MCF7 cells grown in the standard (21%) versus decreased levels (5.0 or 9.9%) of oxygen. The control is set at one fold, which indicates no increased CAT production.

Comparison of the novel plasmid in MCF7 breast cancer cells grown in reduced levels of oxygen. To assess increased levels of CAT production by pCAT-8 contributed by the hypoxia enhancer within the GAPDH sequences, we transfected MCF7 cells and cultured them in standard (21%) or reduced (5.0 or 9.9%) levels of oxygen post transfection. Oxygen levels in tumors have been measured, and tumor hypoxia exists at 1.3% and lower levels of oxygen [18], whereas normal oxygenated tissue has about 5% oxygen. Nevertheless, pCAT-8 produced significantly increased levels of CAT in cells grown in 5.0 or 9.9% oxygen (FIG. 4, P<0.01). Furthermore, MCF7 cells grown in 5.0% oxygen produced slightly higher levels of CAT than cells grown in 9.9% oxygen post transfection with pCAT-8. No significant increase in CAT production was detected in MCF7 cells transfected with pCAT-4 and cultured in 5.0 or 9.9% oxygen post transfection.

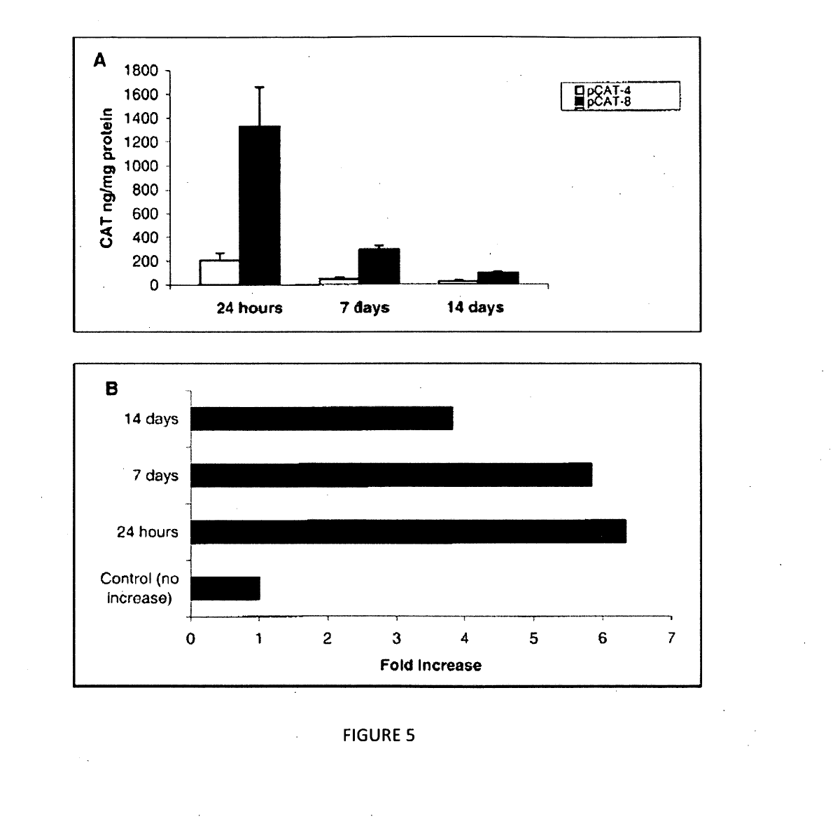

FIG. 5 is a graph of In vitro transfection of MCF7 cells harvested at various time points post transfection using the novel, high-expression plasmid pCAT-8. Our generic in vivo CAT expression plasmid, pCAT-4, was compared to pCAT-8, which contains the optimized CMV promoter-enhancer 5′ to the GAPDH promoter-enhancer. (FIG. 5A) Comparison of pCAT-8 versus pCAT-4 in transfection of MCF7 cells harvested at 24 h, 7 days, or 14 days post transfection. Extruded DOTAP-DNA liposome complexes were used for transfection. CAT production was normalized to protein concentration in the cell extracts and is presented as the mean±SD. (FIG. 5B) The means of the results shown in (FIG. 5A) were compared for fold increased CAT production using pCAT-8 versus pCAT-4 for transfection of MCF7 cells harvested at various time points post transfection. The control is set at onefold, which indicates no increased CAT production.

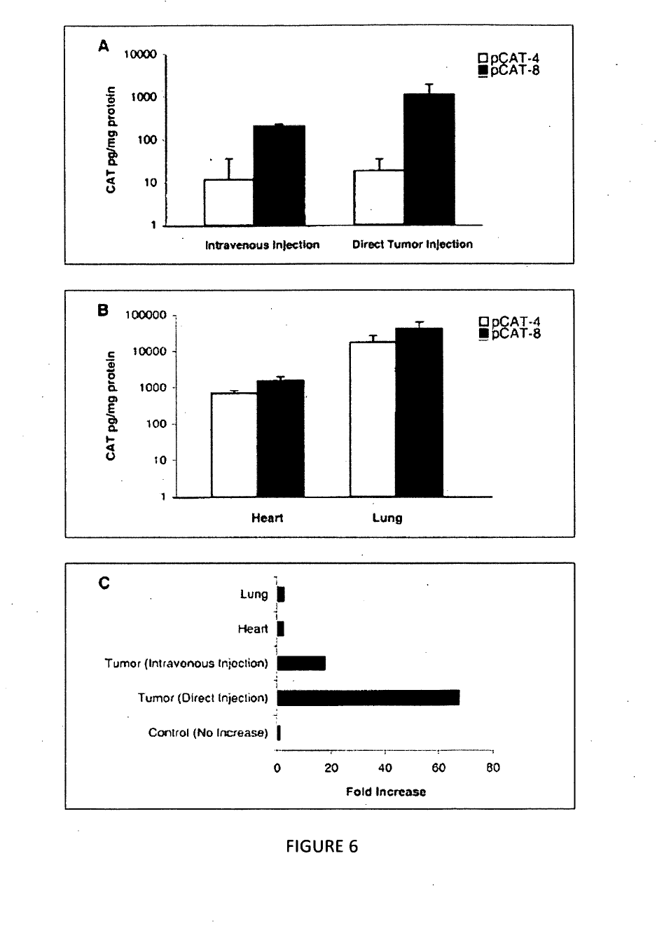

FIG. 6 is a graph of improved gene expression in breast tumors in vivo using pCAT-8. Our generic in vivo CAT expression plasmid, pCAT-4, was compared to pCAT-8, which contains the optimized CMV promoter-enhancer 5′ to the GAPDH promoter-enhancer in tumor-bearing mice. (FIG. 6A) CAT production in MCF7 orthotopic tumors in mice by pCAT-8 or pCAT-4 after intravenous or direct tumor injection of extruded DOTAP:Chol DNA-liposome complexes. CAT production was normalized to protein concentration in the tumor tissue extracts and is presented as the mean±SD. (FIG. 6B) CAT production in the hearts and lungs from the same MCF7 tumor-bearing mice by pCAT-8 or pCAT-4 after intravenous injection of extruded DOTAP:Chol DNA-liposome complexes. CAT production was normalized to protein concentration in the tissue extracts and is presented as the mean±SD. (FIG. 6C) The means of the results shown in (FIG. 6A) and (FIG. 6B) were compared for fold increased CAT production using pCAT-8 versus pCAT-4 for MCF7 tumors after intravenous or direct tumor injections and for heart and lungs from mice injected intravenously. The control is set at one fold, which indicates no increased CAT production.

We performed additional studies to show that either by intravenous or by direct tumor injections. The complexes contained either pCAT-4 or pCAT-8 plasmid DNA. CAT production in the tumors post injection is shown in FIG. 5A. Using pCAT-8 DNA in complexes for direct tumor injections, CAT production increased 67.3-fold (FIG. 6C, P<0.025) compared to production using complexes containing pCAT-4 DNA. The average CAT production increased from 16 to 1076 pg CAT/mg protein in this experiment. In addition, using pCAT-8 DNA in complexes for intravenous injections, CAT production increased 16-fold (FIG. 6C, P<0.025) compared to production using complexes containing pCAT-4 DNA. The average CAT production after intravenous injections increased from 12 to 210 pg CAT/mg protein. These data and data shown in FIG. 4 suggest that further increased CAT production by pCAT-8 is provided by the hypoxia enhancer sequences in response to reduced levels of oxygen. Specifically, pCAT-8 increased CAT production over that of pCAT-4 by 6.2-fold in MCF7 tissue culture cells grown in standard levels of oxygen and up to 67.3-fold in increased CAT production was not due to stabilized gene expression produced by pCAT-8. FIG. 5A shows similar declines in the levels of CAT production in MCF7 cells transfected with pCAT-4 or pCAT-8 out to 14 days post transfection (P<0.01). Therefore, pCAT-8 produced higher levels of gene expression in MCF7 cells due to transcriptional up-regulation of the GAPDH promoter-enhancer and to the response of the hypoxia enhancer to reduced levels of oxygen. Perhaps increased gene expression could be provided, in part, by a potential effect of the GAPDH promoter-enhancer on translation; however, this mechanism was not tested.

Improved gene expression in breast tumors in vivo. Human MCF7 orthotopic breast tumor xenografts were established in female, nude mice (nu/nu) implanted with estradiol tablets. These tumor-bearing mice were injected with extruded DOTAP:Chol DNA-liposome complexes either tumors. Therefore, the hypoxia enhancer within the GAPDH sequences in pCAT-8 mediated an additional 61.1-fold increase in CAT production in MCF7 breast tumors. The heart and lungs were harvested from the identical MCF7 tumor-bearing mice and assayed for CAT production (FIG. 6B). Insignificant increases in CAT production, 2.2- and 2.4-fold (FIG. 6C, P<0.01), were observed in heart and lung tissues, respectively, using pCAT-8 versus pCAT-4 in complexes injected intravenously. Therefore, the GAPDH promoter-enhancer specifically increased CAT production in the MCF7 tumor tissues but not in these normal tissues of the same mice.

Previous work showed that these complexes transfect many cell types, including endothelial cells after intravenous injection [6]. Direct tumor injection would largely bypass delivery to endothelial cells surrounding the tumor and, therefore, would result primarily in the transfection of tumor cells, whereas a greater number of different cell types would be transfected after intravenous delivery in the same tumor-bearing mouse. GAPDH promoter-enhancer sequences do not mediate significantly increased gene expression in endothelial cells (FIG. 6B). Therefore, a 67.3-fold versus a 16-fold increase in CAT production provided by the GAPDH promoter-enhancer in the plasmid would be expected after direct injection of complexes into the tumor.

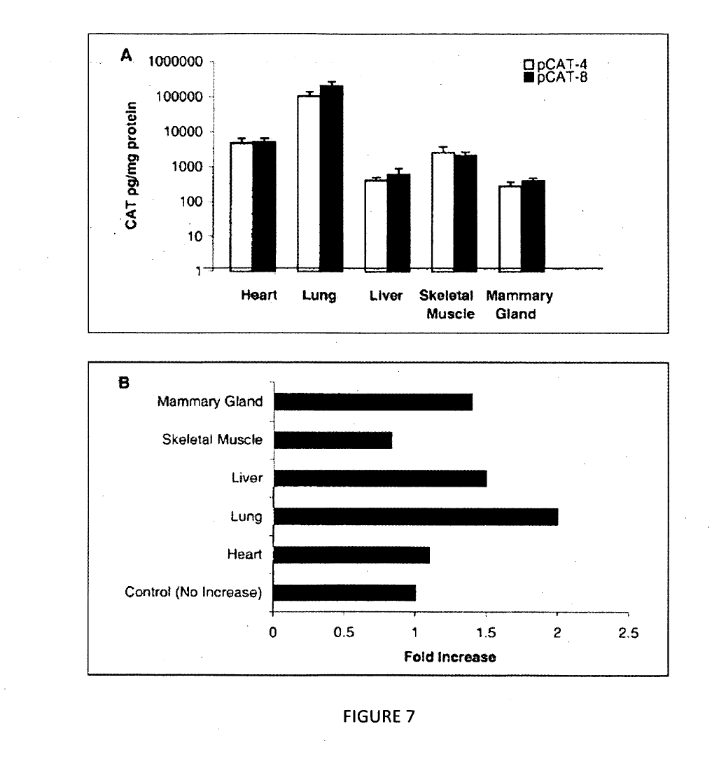

FIG. 7 is a graph comparisons of gene expression in immune-competent, normal mice. Our generic in vivo CAT expression plasmid, pCAT-4, was compared to pCAT-8, which contains the optimized CMV promoter-enhancer 5′ to the GAPDH promoter-enhancer, in female, BALB/c mice. (FIG. 7A) CAT production in the heart, lungs, liver, skeletal muscle, and mammary gland from female BALB/c mice by pCAT-8 or pCAT-4 after intravenous injection of extruded DOTAP:Chol DNA-liposome complexes. CAT production was normalized to protein concentration in the tissue extracts and is presented as the mean±SD. (FIG. 7B) The means of the results shown in (FIG. 7A) were compared for fold increased CAT production using pCAT-8 versus pCAT-4 in tissues from mice injected intravenously. The control is set at onefold, which indicates no increased CAT production.

Comparisons of gene expression in immune-competent, non-tumor-bearing mice. We compared CAT production in normal, BALB/c female mice after intravenous injections of extruded DOTAP:Chol DNA-liposome complexes using pCAT-8 versus pCAT-4 DNA (FIG. 7). Heart, lungs, liver, skeletal muscle, and mammary glands were harvested and assayed for CAT production. CAT production was slightly lower in skeletal muscle using pCAT-8 versus pCAT-4 (FIG. 7B, P<0.01). Furthermore, FIG. 7B shows insignificantly increased CAT production using pCAT-8 versus pCAT-4 for heart (1.1-fold, P<0.01), lung (2.0-fold, P<0.01), liver (1.5-fold, P<0.01), and mammary gland (1.4-fold, P<0.01). Therefore, the GAPDH promoter-enhancer did not increase CAT production in normal tissues, including the mammary gland, further demonstrating specific gene expression in the breast tumor (FIG. 6) and not in normal breast tissue (FIG. 7).

Production of gene expression after transfection is complex and involves more than delivery of DNA into the nucleus. Frequently, delivery of DNA into the nucleus and subsequent gene expression may be poorly correlated. Slight differences in the CMV promoter-enhancers present in plasmids produce different levels of gene expression in similar cell types. For example, FIG. 3 shows about a fourfold difference in CAT production in MCF7 cells using the p4119 CMV promoter-enhancer versus the pVAX CMV promoter-enhancer for transfection in vitro. Our preliminary data from transfection studies using fluorescence-labeled plasmid DNA showed no differences among levels of DNA in the nuclei, suggesting that differences in promoter efficiency rather than delivery of DNA account for the heterogeneity in expression for the eight cell lines analyzed in FIG. 3. Furthermore, our preliminary studies to demonstrate anti-tumor efficacy in MCF7 breast tumor models suggested that greater gene expression was necessary than that produced by plasmids similar to pCAT-4 that lack the CAT gene and contain the proapoptotic gene p53 (pp 53-4). Interestingly, pp 53-4 and similar plasmids demonstrate efficacy in lung tumor models [6]. In addition, our previous work [9] and our current data (FIG. 7) showed good delivery and gene expression of CAT in the mammary gland using the extruded DOTAP:Chol systemic liposomal delivery system. Therefore, the delivery system was not limiting in vivo.

Several investigators have tried to use other nonviral delivery systems that have demonstrated efficacy in animal models for lung cancer to treat breast tumors (N. S. Templeton, personal communication); however, these investigators have failed to show efficacy in breast tumor animal models using the same DNA expression plasmids that they have used to treat lung cancers. They have focused solely on their delivery system as an explanation for the poor results. Perhaps the failure could be caused by both an inefficient delivery system and inefficient gene expression plasmids for breast tumor cells or by the plasmid construct alone. Therefore, we believe that proper plasmid design tailored for gene expression in breast cancer cells and tumors is critical to making progress in nonviral gene therapy for breast cancer.

The issue of plasmid design, however, is far more complex than simply creating custom plasmids for lung cancer versus breast cancer, for example. We showed efficacy in treatment of a syngeneic breast tumor metastasis model after intravenous or direct tumor injections of pEFIRES-based plasmids that express well in PyV MT tumor cells isolated from MMTV polyomavirus middle T transgenic mice [9]. However, FIG. 2 shows that a pEFIRES based plasmid failed to produce gene expression in MCF7 breast tumor cells. Therefore, custom expression plasmids may be required for specific subtypes of breast cancer. Perhaps levels of transcription factors required for plasmid-based gene expression vary among different breast cancer cells, and expression profiling of these breast cancers may predict which patients would respond to plasmids incorporating different promoters and enhancers [10]. In the present studies, we focused on creating a custom gene expression plasmid for MCF7 breast cancer cells and tumors. Our data showed nearly a 70-fold increase in gene expression in MCF7 breast tumors after direct injection and a 16-fold increase in these tumors after intravenous injection using the novel CMV-GAPDH promoter-enhancer (pCAT-8) versus the optimized CMV promoter-enhancer alone (pCAT-4). Significant increases in gene expression were not observed in normal tissues using this plasmid, pCAT-8. Part of this increased gene expression and protein production may be attributable to transcription factors present in MCF7 cells that promote gene expression from the GAPDH promoter. The GAPDH gene encodes a key regulatory enzyme of glycolysis and has been commonly considered a constitutive housekeeping gene. However, the SAGE databases show higher levels of GAPDH transcripts in breast tumor compared to normal cells. Therefore, using a promoter-enhancer identified by the SAGE screening together with an optimal CMV promoter-enhancer we were able to increase gene expression in breast cancer cells.

The bulk of the increased gene expression in breast tumors after in vivo delivery was mediated by the hypoxiaresponsive element [12] within the GAPDH sequences cloned into pCAT-8. Furthermore, these results were supported by our data from transfection of MCF7 cells grown under reduced oxygen culture conditions post transfection. FIG. 4 shows significantly increased CAT production by pCAT-8 in MCF7 cells grown in 5.0 or 9.9% oxygen compared to cells grown in 21% oxygen post transfection. No significant increases in CAT production were detected for cells transfected with pCAT-4 and grown in 5.0 or 9.9% oxygen post transfection. Other hypoxia-responsive elements have been used to increase gene expression within tumors [9-14]. However, hypoxia-responsive elements in viral vectors may not produce as highly elevated levels of gene expression, such as those reported here. Viral vectors encode viral proteins that could act in cis to up-regulate gene expression from promoters, e.g., the cytomegalovirus (CMV) promoter, and therefore are not limited to gene expression produced solely by the host cell transcription factors. Therefore, the basal level of gene expression produced by a viral vector is initially extremely high. Thus, the addition of hypoxiaresponsive elements to viral vectors would not greatly enhance levels of gene expression over the already high basal levels.

Production of high levels of gene expression in target cells using both tissue-specific promoters and tissue-specific enhancers in plasmid vectors has been difficult to achieve. Gene expression is limited to the target cells; however, the overall levels of expression remain relatively low. Therefore, use of hypoxia-responsive elements, particularly those found in the GAPDH promoter region, can provide highly elevated levels of specific gene expression in tumors. This strategy is particularly useful for gene therapy in humans because it does not require the addition of positive or negative regulators or the use of a two-plasmid-based amplification system to provide adequate levels of gene expression. Based on our current studies, we plan to construct and test other custom plasmids for their potential use in cancer gene therapy.

SAGE analyses. The SAGE map xProfiler was used (www.ncbi.nlm.nih.gov/SAGE/sagexpsetup.cgi) to identify highly abundant and selectively expressed transcripts in breast cancer cell lines, DCIS, and normal breast. SAGE Virtual Northern analysis using tools found at www.ncbi.nlm.nih.gov/SAGE/sagevn.cgi and SAGE gene to tag mapping using tools found at www.ncbi.nlm.nih.gov/SAGE/SAGEcid.cgi was also performed.

Plasmid design and construction are shown in Table 2 and detailed under Results. All plasmids were grown under kanamycin selection in DH5α Escherichia coli with the exception of the pEFIRES-based plasmid, pCAT-1, which was grown under ampicillin selection. All plasmids were purified by anionexchange chromatography using the Qiagen Endo-Free Plasmid Giga Kit (Qiagen, Germany). All plasmid pellets were resuspended in 10 mM Tris-HCl, pH 8.0, and stored at −20° C.

Cells were cultured as directed by the ATCC with all cell lines requiring growth in 10% fetal calf serum. For studies comparing MCF7 cells grown in decreased levels of oxygen post transfection, cells were placed into chambers and flushed with hypoxic gas mixtures containing either 5.0 or 9.9% oxygen.

Liposome preparation. Extruded DOTAP and extruded DOTAP:Chol liposomes were prepared as previously described [5]. However, synthetic cholesterol (Sigma, St. Louis, Mo.) was substituted for cholesterol purchased from Avanti Polar Lipids (Alabaster, Ala.) and used at 50:45 DOTAP:Chol. These liposomes are bilamellar invaginated vesicles.

In vitro transfections and CAT assays. Cell lines were cultured in six-well tissue culture clusters to 70% confluency. DNA-liposome complexes were prepared as previously described [5]. Cells were transfected with extruded DOTAP:DNA liposome complexes using 5 μg of DNA per well. Transfections were performed in serum-free medium for 3 h. Six independent in vitro transfections were performed for each data point reported. Enzymelinked immunosorbent assays (ELISAs) were performed using the Roche (Indianapolis, Ind.) CAT ELISA kit. Three control wells for each cell line were transfected with liposomes alone to determine any background levels of CAT production. All CAT protein determinations were corrected for any CAT immunoreactivity detected in the control cells. Protein determinations were performed using the Micro BCA kit (Pierce, Rockford, Ill.). Two-sided Student's t tests were used to determine the P values reported.

In vivo studies. Female nude mice (nu/nu), 5-6 weeks of age, were purchased from Harlan Sprague-Dawley, Inc. (Indianapolis, Ind.). Each mouse was subcutaneously implanted with a 0.125-mg pellet of 1713-estradiol. The following day, MCF7 orthotopic xenografts were established in these mice by injecting 5×10⁶ MCF7 cells suspended in phosphate-buffered saline as previously described [27]. Tumors were grown to approximately 75-100 mm³. Female BALB/c mice, 5-6 weeks of age, were purchased from Harlan Sprague-Dawley, Inc.

In vivo gene delivery. Extruded DOTAP:Chol DNA-liposome complexes were prepared as previously described [5]. For intravenous injections, 100 μA of DNA-liposome complexes containing 50 μg of DNA was injected into the tail vein using a 30-gauge syringe needle and injection over 1 min. For direct tumor injections, 100 μA of DNA-liposome complexes containing 50 μg of DNA was injected into a single site at the center of the tumor using a 30-gauge syringe needle.

Assays for CAT production in tissues. Tissues were harvested and extracts prepared as previously described [5]. ELISAs were performed using the Roche CAT ELISA kit. All CAT protein determinations were corrected for any CAT immunoreactivity detected in the control tissues. Protein determinations were performed using the Micro BCA kit (Pierce). All experimental groups contained 10 mice per group, and controls assessed 5 mice per group. Two-sided Student's t tests were used to determine the P values reported. Control mice were injected with liposomes only. This work was conducted in accordance with the Baylor College of Medicine guidelines using an approved animal protocol.

Therefore, the privileged gene pool may also differ in diseased versus normal cells, and the Keratin 8 gene appears to be aberrantly expressed in several breast cancer cells. Keratin 8 plays an important role in several invasive breast cancers. Keratin 8 is a member of the intermediate filament gene family and is found on the surface of breast cancer cells, including MCF-7 cells. Keratin 8 is the major plasminogen receptor that is required for accelerated activation of cell-associated plasminogen by tissue-type plasminogen activator, which is important for cellular migration, including tumor invasion and metastasis. Keratin 8 has also been used as a diagnostic marker for invasive breast carcinoma and node-positive metastases. Recently, Keratin 8 has also been found in the nucleus and is colocalized with nuclear proteins containing O-linked N-acetylglucosamine residues including Epitope H, hnRNPs, G and A1, c-myc, RNA pol II, and its transcription factors in breast cancer cells and biopsy material from infiltrating ductal breast carcinomas and fibroadenomas. However, the role of nuclear Keratin 8 has not been identified. Interestingly, the glyceraldehyde-3-phosphate dehydrogenase (GAPDH) gene is also a privileged gene in MCF-7 cells and is the seventh most highly expressed gene.

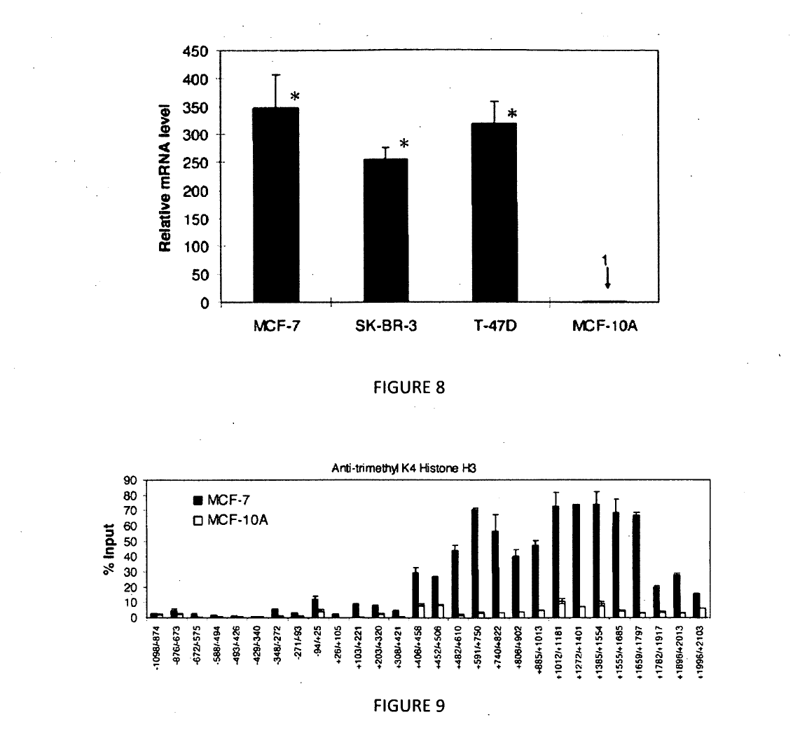

FIG. 8 is a graph of the Keratin 8 mRNA levels in MCF-7, SK-BR-3, T-47D, and MCF-10A cells. The expression levels of Keratin 8 mRNA were quantified by real-time RT-PCR and normalized to the housekeeping gene β-actin. Results are expressed as the ratio relative to Keratin 8 mRNA level in MCF-10A cells. All data are presented as the means±SD of three independent experiments. *p<0.01 versus MCF-10A by Student's t-test. Real-time reverse transcriptase (RT) PCR was performed to quantitatively compare levels of Keratin 8 mRNAs isolated from human breast cancer cell lines including MCF-7, SK-BR-3, and T-47D and the “normal” counterpart breast epithelial cell line, MCF-10A. MCF-10A cells are often used as the near normal control cell line because they are human mammary gland cells that have a normal or near-normal karyotype (23). MCF-7 cells are human breast adenocarcinoma cells that make tumors in immune-deficient mice in response to estrogen. Our results showed that MCF-7 cells had about 350-fold higher levels of Keratin 8 mRNA compared to MCF-10A cells (FIG. 8). T-47D and SK-BR-3 breast cancer cells had approximately 325-fold and 260-fold greater levels of Keratin 8 mRNA, respectively, compared to MCF-10A cells.

Results generated from our ChIP assays using the anti-trimethyl histone H3K4 antibody followed by real-time PCR with primers spanning from 1.1 kb upstream and 2.1 kb downstream of the Keratin 8 start site of transcription are presented in graph of FIG. 9. Numbers along the x-axis indicate the location along the 5′ end of the Keratin 8 gene with “+12” designating the start site of transcription (Table 3). Trimethylation of histone H3K4 on the Keratin 8 gene was compared in MCF-7 versus MCF-10A cells. Histone H3K4 trimethylation was higher in MCF-7 cells and was exceptionally high in regions spanning +406 to +2013, downstream of the Keratin 8 promoter. The highest levels reached 80% over input. Prior to this study, the highest level of histone H3K4 trimethylation reported on an active gene was approximately 20% over input (21).

TABLE 3
5′ Keratin Genomic Region

| | |
|---|---|
| +431 | Intron 1-5′ end |
| +3015 | Intron 1-3′ end |

FIG. 9 is a graph of Histone H3 K4 trimethylation within the Keratin 8 gene in MCF-7 and MCF-10A cells. ChIP assays were performed with antitrimethyl K4 Histone H3. The amount of precipitated DNA was quantified relative to input as described in Materials and Methods. The data are presented as the means±SD of two independent experiments. The initiation site of TATA box is designated at +1.

Furthermore, elevated histone H3K4 trimethylation was found only in short, scattered regions at the start site and sites downstream of the start site of transcription of the active genes examined. No widespread, high levels of histone H3K4 trimethylation were reported. Interestingly, for the Keratin 8 gene in MCF-7 cells, the highest levels of histone H3K4 trimethylation were far downstream of the Keratin 8 gene start site of transcription compared to the start site that showed histone H3K4 trimethylation levels at 10% over input (FIG. 9, from −94/+25).

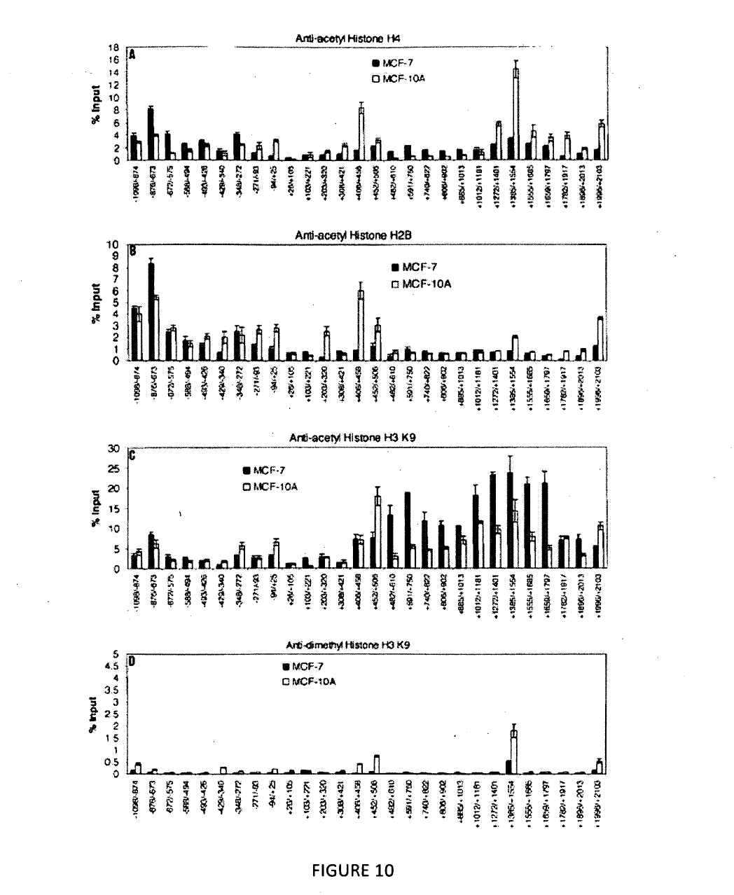

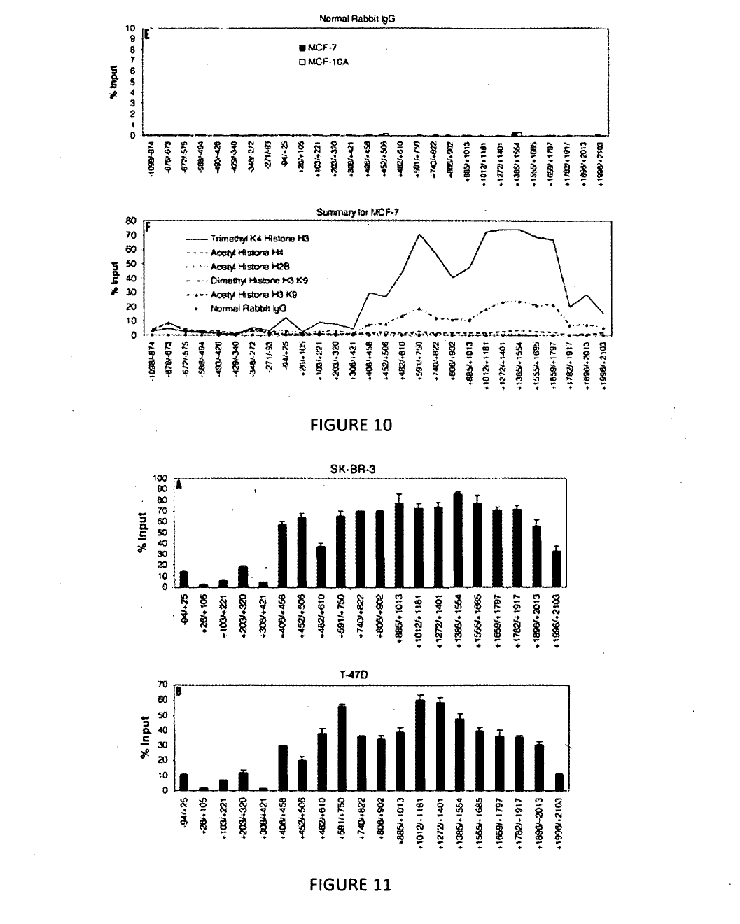

FIGS. 10A to 10F are graphs of the histone modifications within the Keratin 8 gene locus in MCF-7 and MCF-10A cells. ChIP assays were performed with antiacetyl histone H4 (FIG. 10A), anti-acetyl histone H2B (FIG. 10B), anti-acetyl histone H3 K9 (FIG. 10C), or anti-dimethyl histone H3 K9 (FIG. 10D). Normal Rabbit IgG served as the negative control (FIG. 10E). The amount of precipitated DNA was quantified relative to input as described in Materials and Methods. All data are presented as the means±SD of two independent experiments. The initiation site of TATA box is designated at +1. (FIG. 10F) Summary and comparison of the histone modifications examined on the Keratin 8 gene in MCF-7 cells.

Comparisons of Other Histone Modifications on the Keratin 8 Gene in MCF-7 Versus MCF-10A Cells. Histone acetylations (FIG. 10A-C) were examined that are associated with active gene expression and histone H3K9 dimethylation that is associated with gene repression (FIG. 10D). No obvious pattern difference between MCF-7 and MCF-10A Keratin 8 genes was observed for histone H4 acetylation (FIG. 10A). Surprisingly, the MCF-10A Keratin 8 gene showed slightly elevated levels of acetylation at 13 regions upstream and downstream of the promoter, whereas the MCF-7 Keratin 8 gene showed slight elevations in histone H4 acetylation in only four regions. In addition, the MCF-10A Keratin 8 gene showed more regions with minor elevations in histone H2B acetylation compared to that in MCF-7 cells (FIG. 10B). Levels of histone H3K9 acetylation were the highest, reaching 25% over input, for the Keratin 8 gene in MCF-7 cells (FIG. 10C). However, histone H3K9 acetylation in MCF-10A Keratin 8 was only diminished about twofold in eight regions and fourfold in one short region of 138 bp (+1659 to +1797), whereas acetylation levels were significantly higher in MCF-10A Keratin 8 in four regions. Both MCF-7 and MCF-10A Keratin 8 genes showed high levels of histone H3K9 acetylation mainly in the region downstream of the transcription start site, mostly in the first intron, +406 to +2103, the same region that showed even greater levels of trimethylated histone H3K4 in the MCF-7 Keratin 8 gene. Although no obvious pattern difference was detected between the Keratin 8 gene in MCF-7 versus MCF-10A cells with respect to histone H3K9 acetylation, these data suggest that perhaps this acetylation may be necessary but not sufficient to mediate privileged gene expression. Data generated from ChIP assays using the antidimethyl histone H3K9 antibody showed no obvious pattern difference for this histone modification on the Keratin 8 genes in either MCF-7 or MCF-10A cells. These data suggest that histone H3K9 dimethylation is not associated with the low levels of Keratin 8 expression in MCF-10A cells. The negative control data (FIG. 10E) using normal rabbit IgG showed no background in the detection of histone modifications on the Keratin 8 genes in MCF-7 and MCF-10A cells. In summary (FIG. 10F), in the 5′ end of the first intron of Keratin 8 gene in MCF-7 cells, the trimethylated histone H3K4 modification was approximately 3-5-fold, 15-40-fold, and 30-80-fold higher than acetylated histone H3K9, acetylated histone H4, and acetylated histone H2B, respectively. The highest levels of all histone modifications, those above 12% over input, were found only in this intron region and not in the region approximately 1100 bp upstream to 420 bp downstream of the Keratin 8 start site of transcription. At the Keratin 8 promoter, the trimethylated histone H3K4 level was fourfold higher in MCF-7 versus MCF-10A cells, about 12% versus 3% over input. Surprisingly, all histone acetylations were higher at the Keratin 8 gene promoter in MCF-10A versus MCF-7 cells; 3% versus 1% over input for acetylated histone H4 and acetylated histone H2B, and 7% versus 3% over input for acetylated histone H3K9.

FIGS. 11A and 11B are graphs of the Histone H3 K4 trimethylation within the Keratin 8 gene in SK-BR-3 and T-47D cells. ChIP assays were performed with antitrimethyl K4 histone H3 in SK-BR-3 cells (FIG. 11A) or in T-47D cells (FIG. 11B). The data are presented as the means±SD of two independent studies. The initiation site of TATA box is designated at +1. We investigated other breast cancer cell lines, SKBR-3 and T-47D, which showed high levels of Keratin 8 mRNA. The Keratin 8 genes in both cell lines showed highly elevated, widespread histone H3K4 trimethylation in the same first intron region as that in MCF-7, +406 to +2013 (FIG. 11A, 11B). The highest levels of this modification were about 85% and 60% over input for SK-BR-3 and T-47D Keratin 8 genes, respectively. These data support those in FIG. 9 showing that highly elevated levels of Keratin 8 mRNA are associated with widespread, exceptionally high levels of histone H3K4 trimethylation far downstream of the promoter.

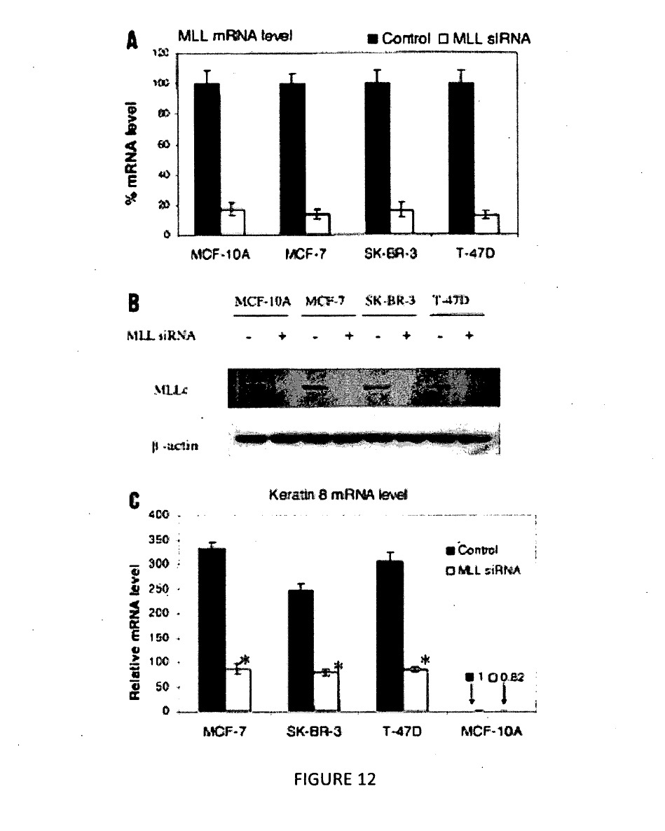

FIGS. 12A to 12C are graphs of the effect of MLL knockdown on Keratin 8 mRNA levels in MCF-7, SK-BR-3, T-47D, and MCF-10A cells. Cells were transfected with MLL siRNA (+) or with control siRNA (−) and harvested 72 h after transfection. (FIG. 12A) Treatment with MLL siRNA results in a decrease of MLL mRNA levels compared to controls. Results are expressed as the percent ratio relative to Keratin 8 mRNA level in control cells. All data are presented as the means±SD of three independent experiments. (FIG. 12B) Treatment with MLL siRNA results in a decrease of MLLc protein levels compared to controls. Whole cell lysate was subjected to Western blotting using anti-MLLc antibody. β-Actin served as the loading control. (FIG. 12C) Analyses of the effect of MLL knockdown on Keratin 8 mRNA levels. Results are expressed as the ratio relative to the Keratin 8 mRNA level in control MCF-10A cells. All data are presented as the means±SD of three independent experiments. *p<0.0001 versus control by Student's t-test.

The C-terminal SET domain of the MLL protein family is a specific histone methyltransferase that methylates only histone H3K4. Furthermore, MLL associates with some transcriptionally active genes and regulates gene expression at the stage of elongation. Most likely, MLL travels with RNA pol II during transcription elongation. To quantify the contribution of the histone H3K4 trimethylation to the expression of the Keratin 8 gene in MCF-7 cells, we performed knockdown experiments of MLL using siRNAs. All cell lines studied had similar levels of MLL mRNA and protein. In all cell lines, MLL mRNA levels were decreased between 87% and 83% (FIG. 12A) in response to transfection with MLL siRNA. MLL was knocked down by 86% in MCF-7 cells. These knockdown results were further supported by Western blot analyses to detect MLL protein in all cell lines transfected with MLL siRNA (+) or with unrelated control siRNA (−) (FIG. 12B). Our results also showed large decreases in Keratin 8 mRNA levels in MCF-7, SK-BR-3, and T-47D cells associated with MLL knockdown (FIG. 12C). These decreases were detected and quantified by realtime RT-PCR on mRNA from cell lines transfected with MLL siRNA or control siRNA. Keratin 8 mRNA levels were decreased by 74%, 68%, and 72% in MCF-7, SK-BR-3, and T-47D cells, respectively. Typically, siRNA does not knockdown gene expression by 100%. Therefore, if MLL siRNA knockdown in MCF-7 cells was 100% effective, we predict that the Keratin 8 mRNA levels would be reduced by 86%.

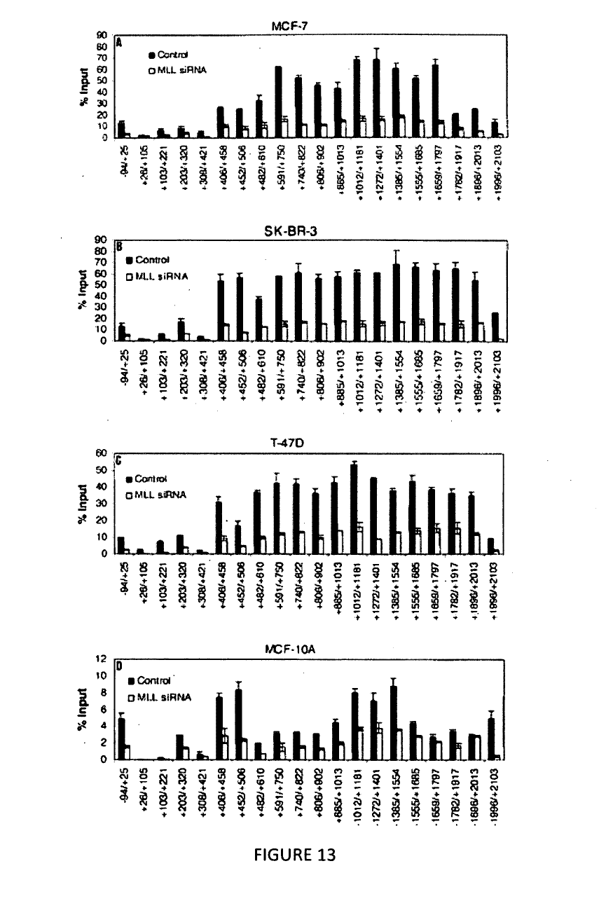

FIGS. 13A to 13D are graphs that show the effects of MLL knockdown on Histone H3 K4 trimethylation within the Keratin 8 gene. MCF-7 (FIG. 13A), SK-BR-3 (FIG. 13B), T-47D (FIG. 13C), and MCF-10A (FIG. 13D) cells were transfected with MLL siRNA or unrelated control siRNA and used for ChIP assays 72 h after transfection. The amount of precipitated DNA was quantified relative to input as described in Materials and Methods. All data are presented as the means±SD of two independent experiments. The initiation site of TATA box is designated at +1.

Knockdown of MLL Decreases Histone H3K4 Trimethylation of the Keratin 8 Gene in all Cell Lines. To support data presented in FIGS. 13A to 13D, we confirmed that knockdown of MLL did significantly reduce histone H3K4 trimethylation levels in all Keratin 8 regions in all cell lines (FIG. 13A-D). This histone modification was obviously most greatly reduced in the region from +406 to +2103 where the histone H3K4 trimethylation levels are extraordinarily high in cell lines treated with control siRNA. DISCUSSION We investigated and extensively quantified the role of histone modifications in privileged gene expression. We used the Keratin 8 gene as a model system, which is the gene that has the highest sustained expression in MCF-7 cells. We found extremely high levels of histone H3K4 trimethylation throughout 1.6 kb of the 5′ end of the first intron located about 400 bp downstream of the Keratin 8 promoter. Histone acetylations, commonly associated with active gene expression, and histone H3K9 dimethylation associated with gene repression, showed no outstanding differences between the Keratin 8 genes in MCF-7 cells versus the control MCF-10A cells that have about 350-fold reduced levels of Keratin 8 mRNA. Through quantitative MLL knockdown experiments, we estimated that approximately 86% of the Keratin 8 privileged gene expression observed in MCF-7 cells is contributed by histone H3K4 trimethylation. MLL regulates gene expression at the elongation stage by an unknown mechanism, and experiments performed in null MLL knockout cells showed that RNA pol II seemed to be stalled on MLL responsive genes. Although these published studies suggest that sequence-specific transcription factors may exist that recruit MLL to MLL target genes, our present work comparing MCF-10A to human breast cancer cells demonstrates that sequence specificity is not sufficient. Instead, recruitment of MLL to genes is also cell type specific, and, therefore, is not dependent on gene-specific sequences alone. Further work on the MLL complex found at the Keratin 8 gene in MCF-7 cells could substantially contribute to understanding how histone H3K4 trimethylation contributes to the most highly efficient transcription elongation. MLL has been shown to specifically associate with only a few transcriptionally active promoters. Furthermore, somewhat elevated levels of histone H3K4 trimethylation have been found associated with active gene expression, up to approximately 20% over input, in short regions around and downstream of promoters for genes that are turned on and off during development. We have found further elevated levels of histone H3K4 trimethylation, up to 80% over input, in a long stretch downstream of the promoter for the Keratin 8 gene in MCF-7 cells. Work from other groups involving tightly focused ChIP assays have demonstrated loci of histone H3K4 methylation associated with active gene expression and separated by boundaries to prevent gene inactivation associated with histone H3K9 methylation. In the chicken β-globin gene, insulator elements are the barriers that separate this gene locus from neighboring genes, and thus are not affected by surrounding chromosomal silencing. The insulator elements perform this function by constitutively recruiting histone H3K4 methylation and histone acetylation in order to prevent propagation of condensed chromatin. In fission yeast, the mating-type locus consists of heterochromatic and euchromatic regions that are separated by boundary elements consisting of two inverted repeats. Histone H3K9 methylation and the Swi6 protein are localized to repressed gene expression in heterochromatin. Conversely, histone H3K4 methylation is present on active genes in the euchromatic region. Our identification of highly elevated levels of histone H3K4 trimethylation downstream of the Keratin 8 promoter supports transcription elongation as the key contributor to the ability of privileged genes to produce the highest levels of gene expression. Further understanding of highly efficient transcription elongation would be useful. Perhaps the Keratin 8 gene in MCF-7 cells produces a far greater number of full-length transcripts than all other genes in those cells. Production of far more full-length transcripts could be mediated by factors that bind to or associate with trimethylated histone H3K4 downstream of the promoter. These factors could assist in RNA pol II transcription elongation and ensure completion of mRNA transcription. Higher levels of transcription reinitiation at the Keratin 8 promoter in MCF-7 cells may also be enhanced by histone H3K4 trimethylation and could contribute to privileged gene expression. No outstanding pattern differences in histone modifications were noted upstream of the Keratin 8 promoters in MCF-7 versus MCF-10A. Apparently, a higher order of histone modifications exists within this model system in which histone H3K4 trimethylation far downstream of the promoter dictates the vast majority of privileged gene expression.

It is contemplated that any embodiment discussed in this specification can be implemented with respect to any method, kit, reagent, or composition of the invention, and vice versa. Furthermore, compositions of the invention can be used to achieve methods of the invention.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All publications and patent applications mentioned in the specification are indicative of the level of skill of those skilled in the art to which this invention pertains. All publications and patent applications are herein incorporated by reference to the same extent as if each individual publication or patent application was specifically and individually indicated to be incorporated by reference.

The use of the word “a” or “an” when used in conjunction with the term “comprising” in the claims and/or the specification may mean “one,” but it is also consistent with the meaning of “one or more,” “at least one,” and “one or more than one.” The use of the term “or” in the claims is used to mean “and/or” unless explicitly indicated to refer to alternatives only or the alternatives are mutually exclusive, although the disclosure supports a definition that refers to only alternatives and “and/or.” Throughout this application, the term “about” is used to indicate that a value includes the inherent variation of error for the device, the method being employed to determine the value, or the variation that exists among the study subjects.

As used in this specification and claim(s), the words “comprising” (and any form of comprising, such as “comprise” and “comprises”), “having” (and any form of having, such as “have” and “has”), “including” (and any form of including, such as “includes” and “include”) or “containing” (and any form of containing, such as “contains” and “contain”) are inclusive or open-ended and do not exclude additional, unrecited elements or method steps. As used herein, the phrase “consisting essentially of” limits the scope of a claim to the specified materials or steps and those that do not materially affect the basic and novel characteristic(s) of the claimed invention. As used herein, the phrase “consisting of” excludes any element, step, or ingredient not specified in the claim except for, e.g., impurities ordinarily associated with the element or limitation.

The term “or combinations thereof” as used herein refers to all permutations and combinations of the listed items preceding the term. For example, “A, B, C, or combinations thereof” is intended to include at least one of: A, B, C, AB, AC, BC, or ABC, and if order is important in a particular context, also BA, CA, CB, CBA, BCA, ACB, BAC, or CAB. Continuing with this example, expressly included are combinations that contain repeats of one or more item or term, such as BB, AAA, MB, BBC, AAABCCCC, CBBAAA, CABABB, and so forth. The skilled artisan will understand that typically there is no limit on the number of items or terms in any combination, unless otherwise apparent from the context.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations may be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

1. Templeton, N. S. (2001). Developments in liposomal gene delivery systems. Expert Opin. Biol. Ther. 1:1-4.
2. Handumrongkul, C., Zhong, W., and Debs, R. J. (2002). Distinct sets of cellular genes control the expression of transfected, nuclear-localized genes. Mol. Ther. 5: 186-194.
3. Danesi, R., DeBraud, F., Fogli, S., DiPaolo, A., and Del Tacca, M. (2001). Pharmacogenetic determinants of anti-cancer drug activity and toxicity. Trends Pharmacol. Sci. 22: 420-426.
4. Johnstone, R. W., Ruefli, A. A., and Lowe, S. W. (2002). Apoptosis: A link between cancer genetics and chemotherapy. Cell 108: 153-164.
5. Templeton, N. S., et al. (1997). Improved DNA: Liposome complexes for increased systemic delivery and gene expression. Nat. Biotech. 15: 647-652.
6. Ramesh, R., et al. (2001). Successful treatment of primary and disseminated human lung cancers by systemic delivery of tumor suppressor genes using an improved liposome vector. Mol. Ther. 3: 337-350.
7. Tirone, T. A., Fagan, S. P., Templeton, N. S., Wang, X. P., and Brunicardi, F. C. (2001). Insulinoma induced hypoglycemic death in mice is prevented with beta cell specific gene therapy. Ann. Surg. 233: 603-611.
8. Yotnda, P., et al. (2002). Bilamellar cationic liposomes protect adenovectors from preexisting humoral immune responses. Mol. Ther. 5: 233-241.
9. Shi, H. Y., Liang, R., Templeton, N. S., and Zhang, M. (2002). Inhibition of breast tumor progression by systemic delivery of the maspin gene in a syngeneic tumor model. Mol. Ther. 5: 755-761.
10. Ince, T. A., and Weinberg, R. A. (2002). Functional genomics and the breast cancer problem. Cancer Cell 1: 5-17.
11. Velculescu, V. E., Zhang, L., Vogelstein, B., and Kinzler, K. W. (1995). Serial analysis of gene expression. Science 270: 484-487.
12. Graven, K. K., Yu, Q., Pan, D., Roncarati, J. S., and Farber, H. W. (1999). Identification of an oxygen responsive enhancer element in the glyceraldehyde-3-phosphate dehydrogenase gene. Biochim. Biophys. Acta 1447: 208-218.
13. Lash, A. E., et al. (2000). SAGEmap: A public gene expression resource. Genome Res. 10: 1051-1060.
14. Lal, A., et al. (1999). A public database for gene expression in human cancers. Cancer Res. 59: 5403-5407.
15. Aki, T., Yanagisawa, S., and Akanuma, H. (1997). Identification and characterization of positive regulatory elements in the human glyceraldehyde 3-phosphate dehydrogenase gene promoter. J. Biochem. 122: 271-278.
16. Zhu, N., Liggitt, D., Liu, Y., and Debs, R. (1993). Systemic gene expression after intravenous DNA delivery in adult mice. Science 261: 209-211.
17. Liu, Y., et al. (1995). Cationic liposome-mediated intravenous gene delivery. J. Biol. Chem. 270: 24864-24870.
18. Vaupel, P. (1996). Is there a critical tissue oxygen tension for bioenergetic status and cellular pH regulation in solid tumors? Experientia 52: 464-468.
19. Cao, Y. J., Shibata, T., and Rainov, N. G. (2001). Hypoxia-inducible transgene expression in differential human NT2N neurons: A cell culture model for gene therapy of postischemic neuronal loss. Gene Ther. 8: 1357-1362.
20. Ruan, H., et al. (2001). A hypoxia-regulated adeno-associated virus vector for cancerspecific gene therapy. Neoplasia 3: 255-263.
21. Ido, A., et al. (2001). Gene therapy targeting for hepatocellular carcinoma: Selective and enhanced suicide gene expression regulated by a hypoxia-inducible enhancer linked to a human alpha-fetoprotein promoter. Cancer Res. 61: 3016-3021.
22. Dachs, G. U., Coralli, C., Hart, S. L., and Tozer, G. M. (2000). Gene delivery to hypoxic cells in vitro. Br. J. Cancer 83: 662-667.23. Modlich, U., Pugh, C. W., and Bicknell, R. (2000). Increasing endothelial cell specific expression by the use of heterologous hypoxic and cytokine-inducible enhancers. Gene Ther. 7: 896-902.
24. Shibata, T., Giaccia, A. J., and Brown, J. M. (2000). Development of a hypoxiaresponsive vector for tumor-specific gene therapy. Gene Ther. 7: 493-498.
25. Benz, C. C., et al. (1992). Estrogen-dependent, tamoxifen-resistant tumorigenic growth of MCF-7 cells transfected with HER2/neu. Breast Cancer Res. Treat. 24: 85-95.
26. Gazdar, A. F., et al. (1998). Characterization of paired tumor and non-tumor cell lines established from patients with breast cancer. Int. J. Cancer 78: 766-774.
27. Boland, A., et al. (2000). Adenovirus-mediated transfer of the thyroid sodium/iodide symporter gene into tumors for a targeted radiotherapy. Cancer Res. 60: 3484-3492.

What is claimed is:

1. A modified histone octamer for gene delivery comprising:
a modified histone core comprising an histone octamer of H2A, H2B, H3 and H4 with one or more modifications of the histone octamer;
an expression cassette complexed to the histone octamer comprising a glyceraldehyde-3-phosphate dehydrogenase (GAPDH)-cytomegalovirus (CMV) promoter-enhancer sequence and one or more genes; and
a bi-lamellar liposome encapsulant that encapsulates the expression cassette for increased post-transfection transcription.

2. The composition of claim 1, wherein the modified histone octamer core comprises two H2A/H2B dimers and one H3/H4 tetramer.

3. The composition of claim 1, wherein the modified histone octamer core comprises acetylation of the histones and methylation of histone H3 lysine 4.

4. The composition of claim 1, wherein the modified histone octamer core comprises trimethylation of histone H3 lysine 4.

5. The composition of claim 1, further comprising the addition of one or more HDAC inhibitors to treat a cancer cell by increasing the expression levels of genes required to induce cell cycle arrest and/or apoptosis.

6. The composition of claim 1, wherein the HDAC inhibitors comprise valproic acid, vorinostat, or a combination thereof.

7. The composition of claim 1, wherein the bi-lamellar liposome encapsulant comprises dioleoylphosphatidylethanolamine, dilauroylphosphatidylcholine or a combination thereof.

8. The composition of claim 1, wherein the bi-lamellar liposome encapsulant comprises N-1-(2,3dioleyloxy)propyl-N,N,N-trimethylammonium chloride (DOTMA), cholesterol, phosphatidylcholines, or phosphatidylserines, 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), Stearylamine, 1,2-dioleoyl-3-dimethyl-hydroxyethyl ammonium bromide, (1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), O,O′-didodecyl-N-p-(2-trimethylammonioethyloxy)benzoyl-N,N,N-tri-methylammonium chloride, Lipospermine, DC-Chol (3a-N-(N′,N″-dimethylaminoethane)carbonylcholesterol), lipopoly(L-lysine), and mixtures and combinations thereof.

9. A modified reconstituted histone octamer for gene therapy composition comprising:
an isolated, modified histone octamer core comprising H2A, H2B, H3 and H4;
an expression cassette complexed to the modified histone octamer comprising an promoter-enhancer and a DNA sequence; and
a liposome that encapsulates expression cassette.

10. The composition of claim 9, wherein the modified histone octamer core comprises two H2A/H2B dimers and one H3/H4 tetramer.

11. The composition of claim 9, wherein the modified histone octamer core comprises acetylation of the histones and methylation of histone H3 lysine 4.

12. The composition of claim 9, wherein the modified histone octamer core comprises trimethylation of histone H3 lysine 4.

13. The composition of claim 9, wherein the expression cassette comprises a glyceraldehyde-3-phosphate dehydrogenase (GAPDH) sequence and a cytomegalovirus (CMV) sequence.

14. The composition of claim 9, wherein the DNA sequence comprises a therapeutic gene.

15. The composition of claim 9, wherein the DNA sequence comprises plasmids for increased transcription of full-length transcripts post-transfection.

16. The composition of claim 9, wherein the liposome comprises a bi-lamellar invaginated liposome

17. The composition of claim 9, further comprising the addition of one or more HDAC inhibitors to treat a cancer cell by increasing the expression levels of genes required to induce cell cycle arrest and/or apoptosis.

18. The composition of claim 9, wherein the HDAC inhibitors comprise valproic acid, vorinostat, or a combination thereof.

19. The composition of claim 9, wherein the bi-lamellar liposome encapsulant comprises dioleoylphosphatidylethanolamine, dilauroylphosphatidylcholine or a combination thereof.

20. The composition of claim 9, wherein the bi-lamellar liposome encapsulant comprises N-1-(2,3dioleyloxy)propyl-N,N,N-trimethylammonium chloride (DOTMA), cholesterol, phosphatidylcholines, or phosphatidylserines, 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), Stearylamine, 1,2-dioleoyl-3-dimethyl-hydroxyethyl ammonium bromide, (1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), O,O′-didodecyl-N-p-(2-trimethylammonioethyloxy)benzoyl-N,N,N-tri-methylammonium chloride, Lipospermine, DC-Chol (3a-N-(N′,N″-dimethylaminoethane)carbonylcholesterol), lipopoly(L-lysine), and mixtures and combinations thereof.

21. A method of treating a patient having cancer comprising the steps of:
selecting a patient having one or more cancerous cells; and
providing a therapeutic amount of a pharmaceutical composition comprising an isolated, reconstituted and modified histone octamer for gene delivery having a modified histone core comprising an histone octamer of H2A, H2B, H3 and H4 with one or more modifications of the histone octamer, an expression cassette complexed to the histone octamer comprising a glyceraldehyde-3-phosphate dehydrogenase (GAPDH)-cytomegalovirus (CMV) promoter-enhancer sequence and one or more genes, a bi-lamellar liposome encapsulant that encapsulates the expression cassette for increased post-transfection transcription and a pharmaceutical carrier.

22. The method of claim 21, wherein the modified histone octamer core comprises two H2A/H2B dimers and one H3/H4 tetramer.

23. The method of claim 21, wherein the modified histone octamer core comprises acetylation of the histones and methylation of histone H3 lysine 4.

24. The method of claim 21, wherein the modified histone octamer core comprises trimethylation of histone H3 lysine 4.

25. The method of claim 21, further comprising the addition of one or more HDAC inhibitors to treat a cancer cell by increasing the expression levels of genes required to induce cell cycle arrest and/or apoptosis.

26. The method of claim 21, wherein the HDAC inhibitors comprise valproic acid, vorinostat, or a combination thereof.

27. The method of claim 21, wherein the bi-lamellar liposome encapsulant comprises dioleoylphosphatidylethanolamine, dilauroylphosphatidylcholine or a combination thereof.

28. The method of claim 21, wherein the bi-lamellar liposome encapsulant comprises N-1-(2,3dioleyloxy)propyl-N,N,N-trimethylammonium chloride (DOTMA), cholesterol, phosphatidylcholines, or phosphatidylserines, 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), Stearylamine, 1,2-dioleoyl-3-dimethyl-hydroxyethyl ammonium bromide, (1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), O,O′-didodecyl-N-p-(2-trimethylammonioethyloxy)benzoyl-N,N,N-tri-methylammonium chloride, Lipospermine, DC-Chol (3a-N-(N′,N″-dimethylaminoethane)carbonylcholesterol), lipopoly(L-lysine), and mixtures and combinations thereof.

29. A process for delivering a nucleic acid to a mammalian cell comprising the steps of:
delivering a therapeutic amount of a pharmaceutical composition to a mammalian cell, wherein the pharmaceutical composition comprises a reconstituted, modified histone octamer for gene delivery having a modified histone core comprising an histone octamer of H2A, H2B, H3 and H4 with one or more modifications of the histone octamer, an expression cassette complexed to the histone octamer comprising a glyceraldehyde-3-phosphate dehydrogenase (GAPDH)-cytomegalovirus (CMV) promoter-enhancer sequence and one or more genes, a bi-lamellar liposome encapsulant that encapsulates the expression cassette for increased post-transfection transcription and a pharmaceutical carrier.

30. The method of claim 29, wherein the modified histone octamer core comprises two H2A/H2B dimers and one H3/H4 tetramer.

31. The method of claim 29, wherein the modified histone octamer core comprises acetylation of the histones and methylation of histone H3 lysine 4.

32. The method of claim 29, further comprising the modified histone octamer core comprises trimethylation of histone H3 lysine 4.

33. The method of claim 29, wherein the addition of one or more HDAC inhibitors to treat a cancer cell by increasing the expression levels of genes required to induce cell cycle arrest and/or apoptosis.

34. The method of claim 29, wherein the HDAC inhibitors comprise valproic acid, vorinostat, or a combination thereof.

35. The method of claim 29, wherein the bi-lamellar liposome encapsulant comprises dioleoylphosphatidylethanolamine, dilauroylphosphatidylcholine or a combination thereof.

36. The method of claim 29, wherein the bi-lamellar liposome encapsulant comprises N-1-(2,3dioleyloxy)propyl-N,N,N-trimethylammonium chloride (DOTMA), cholesterol, phosphatidylcholines, or phosphatidylserines, 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), Stearylamine, 1,2-dioleoyl-3-dimethyl-hydroxyethyl ammonium bromide, (1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), O,O′-didodecyl-N-p-(2-trimethylammonioethyloxy)benzoyl-N,N,N-tri-methylammonium chloride, Lipospermine, DC-Chol (3a-N-(N′,N″-dimethylaminoethane)carbonylcholesterol), lipopoly(L-lysine), and mixtures and combinations thereof.

37. A method to regulate expression of a nucleic acid sequence of interest comprising the steps of:
providing a therapeutic amount of a pharmaceutical composition comprising an isolated, reconstituted and modified histone octamer for gene delivery having a modified histone core comprising an histone octamer of H2A, H2B, H3 and H4 with one or more modifications of the histone octamer, an expression cassette complexed to the histone octamer comprising a glyceraldehyde-3-phosphate dehydrogenase (GAPDH)-cytomegalovirus (CMV) promoter-enhancer sequence and one or more genes, a bi-lamellar liposome encapsulant that encapsulates the expression cassette for increased post-transfection transcription and a pharmaceutical carrier.

38. The method of claim 37, wherein the modified histone octamer core comprises two H2A/H2B dimers and one H3/H4 tetramer.

39. The method of claim 37, wherein the modified histone octamer core comprises acetylation of the histones and methylation of histone H3 lysine 4.

40. The method of claim 37, wherein the modified histone octamer core comprises trimethylation of histone H3 lysine 4.

41. The method of claim 37, further comprising the addition of one or more HDAC inhibitors to treat a cancer cell by increasing the expression levels of genes required to induce cell cycle arrest and/or apoptosis.

42. The method of claim 37, wherein the HDAC inhibitors comprise valproic acid, vorinostat, or a combination thereof.

43. The method of claim 37, wherein the bi-lamellar liposome encapsulant comprises dioleoylphosphatidylethanolamine, dilauroylphosphatidylcholine or a combination thereof.

44. The method of claim 37, wherein the bi-lamellar liposome encapsulant comprises N-1-(2,3dioleyloxy)propyl-N,N,N-trimethylammonium chloride (DOTMA), cholesterol, phosphatidylcholines, or phosphatidylserines, 1,2-bis(oleoyloxy)-3-(4′-trimethylammonio)propane (DOTAP), 1,2-dioleoyl-3-(4′-trimethylammonio)butanoyl-sn-glycerol (DOTB), 1,2-dioleoyl-3-succinyl-sn-glycerol choline ester (DOSC), cholesteryl (4′-trimethylammonio)butanoate (ChoTB), cetyltrimethylammonium bromide (CTAB), Stearylamine, 1,2-dioleoyl-3-dimethyl-hydroxyethyl ammonium bromide, (1,2-dioleyloxypropyl-3-dimethyl-hydroxyethyl ammonium bromide (DORIE), 1,2-dimyristyloxypropyl-3-dimethyl-hydroxyethyl ammonium bromide (DMRIE), O,O′-didodecyl-N-p-(2-trimethylammonioethyloxy)benzoyl-N,N,N-tri-methylammonium chloride, Lipospermine, DC-Chol (3a-N-(N′,N″-dimethylaminoethane)carbonylcholesterol), lipopoly(L-lysine), and mixtures and combinations thereof.

45. A method of forming a reconstituted modified histone octamer for gene delivery comprising the steps of:
reconstituting a modified histone core comprising an histone octamer of H2A, H2B, H3 and H4 with one or more modifications of the histone octamer;
complexing an expression cassette with the histone octamer, wherein the expression cassette comprises a glyceraldehyde-3-phosphate dehydrogenase (GAPDH)-cytomegalovirus (CMV) promoter-enhancer sequence and one or more genes; and
encapsulating the expression cassette in a bi-lamellar liposome encapsulant to increase post-transfection transcription.
